(19) United States
(12) Patent Application Publication
(10) Pub. No.: US 2011/0251684 A1
(43) Pub. Date: Oct. 13, 2011

(54) DEVICES, SYSTEMS, AND METHODS FOR RESHAPING A HEART VALVE ANNULUS

(75) Inventors: DAVID A. RAHDERT, San Francisco, CA (US); Timothy R. Machold, Moss Beach, CA (US); Robert T. Chang, Belmont, CA (US)

(73) Assignee: MVRx, Inc., Moss Beach, CA (US)

(21) Appl. No.: 13/164,115

(22) Filed: Jun. 20, 2011

Related U.S. Application Data
(63) Continuation of application No. 12/284,199, filed on Sep. 19, 2008.
Application No. 12/284,199 is a continuation-in-part of application No. 11/389,819, filed on Mar. 27, 2006, now abandoned.
Application No. 11/389,819 is a continuation-in-part of application No. 11/255,663, filed on Oct. 21, 2005.
Application No. 11/255,663 is a continuation-in-part of application No. 11/089,940, filed on Mar. 25, 2005, now Pat. No. 7,691,144.
Application No. 11/089,940 is a continuation-in-part of application No. 10/894,433, filed on Jul. 19, 2004.
Application No. 10/894,433 is a continuation-in-part of application No. 10/846,850, filed on May 14, 2004.
Application No. 10/846,850 is a continuation-in-part of application No. 11/903,407, filed on Sep. 21, 2007, now abandoned.

(51) Int. Cl. A61F 2/24

(52) U.S. Cl. 623/2.11; 623/2.1

(57) ABSTRACT

Implants or systems of implants and methods apply an upward and inward force producing a minor axis force projection vector within or across the left atrium, which allow mitral valve leaflets to better coapt. The implants or systems of implants and methods make possible rapid deployment, facile endovascular delivery, and full intra-atrial retrievability. The implants or systems of implants and methods also make use of strong fluoroscopic landmarks. The implants or systems of implants and methods make use of an adjustable implant. The implants or systems of implants and methods may also utilize a bridge stop to secure the implant, and the methods of implantation employ various tools.

### RELATED APPLICATIONS

This application is a continuation of co-pending U.S. patent application Ser. No. 12/284,199 filed 19 Sep. 2008, which is a continuation-in-part of co-pending U.S. patent application Ser. No. 11/903,407, filed 21 Sep. 2007, end entitled “Devices, Systems and Methods for Reshaping a Heart Valve Annulus, Including the use of a Bridge Implant having an Adjustable Bridge Stop,” which is incorporated herein by reference.

This application is a continuation-in-part of co-pending U.S. patent application Ser. No. 11/389,819, filed 27 Mar. 2006, end entitled “Devices, Systems and Methods for Reshaping a Heart Valve Annulus,” which is incorporated herein by reference.

This application is a continuation-in-part of co-pending U.S. patent application Ser. No. 11/255,663, filed 21 Oct. 2005, and entitled “Devices, Systems, and Methods for Reshaping a Heart Valve Annulus, Including the Use of a Bridge Implant Having an Adjustable Bridge Stop” which is incorporated herein by reference.

This application is a continuation-in-part of co-pending U.S. patent application Ser. No. 11/089,940, filed 25 Mar. 2005, end entitled “Devices, Systems, and Methods for Reshaping a Heart Valve Annulus,” which is incorporated herein by reference.

This application is a continuation-in-part of co-pending U.S. patent application Ser. No. 10/894,433, filed 19 Jul., 2004, and entitled “Devices, Systems, and Methods for Reshaping a Heart Valve Annulus.” which is incorporated herein by reference.

This application is a continuation-in-part of co-pending U.S. patent application Ser. No. 10/846,850, filed 14 May 2004, and entitled “Devices, Systems, and Methods for Reshaping a Heart Valve Annulus,” which is incorporated herein by reference.

### FIELD OF THE INVENTION

The invention is directed to devices, systems, and methods for improving the function of a heart valve, e.g., in the treatment of mitral valve regurgitation.

### BACKGROUND OF THE INVENTION

### I. The Anatomy of a Healthy Heart

The heart (see FIG. 1) is slightly larger than a clenched fist. It is a double (left and right side), self-adjusting muscular pump, the parts of which work in unison to propel blood to all parts of the body. The right side of the heart receives poorly oxygenated (“venous”) blood from the body from the superior vena cava and inferior vena cava and pumps it through the pulmonary artery to the lungs for oxygenation. The left side receives well-oxygenation (“arterial”) blood from the lungs through the pulmonary veins and pumps it into the aorta for distribution to the body.

The heart has four chambers, two on each side—the right and left atria, and the right and left ventricles. The atriums are the blood-receiving chambers, which pump blood into the ventricles. The ventricles are the blood-discharging chambers. A wall composed of fibrous and muscular parts, called the interatrial septum separates the right and left atriums (see FIGS. 2 to 4). The fibrous interatrial septum is, compared to the more friable muscle tissue of the heart, a more materially strong tissue structure in its own extent in the heart. An anatomic landmark on the interatrial septum is an oval, thumbprint sized depression called the oval fossa, or fossa ovalis (shown in FIGS. 4 and 6), which is a remnant of the oval foramen and its valve in the fetus. It is free of any vital structures such as valve structure, blood vessels and conduction pathways. Together with its inherent fibrous structure and surrounding fibrous ridge which makes it identifiable by angiographic techniques, the fossa ovalis is the favored site for trans-septal diagnostic and therapeutic procedures from the right into the left heart. Before birth, oxygenated blood from the placenta was directed through the oval foramen into the left atrium, and after birth the oval foramen closes.

The synchronous pumping actions of the left and right sides of the heart constitute the cardiac cycle. The cycle begins with a period of ventricular relaxation, called ventricular diastole. The cycle ends with a period of ventricular contraction, called ventricular systole.

The heart has four valves (see FIGS. 2 and 3) that ensure that blood does not flow in the wrong direction during the cardiac cycle; that is, to ensure that the blood does not back flow from the ventricles into the corresponding atria, or back flow from the arteries into the corresponding ventricles. The valve between the left atrium and the left ventricle is the mitral valve. The valve between the right atrium and the right ventricle is the tricuspid valve. The pulmonary valve is at the opening of the pulmonary artery. The aortic valve is at the opening of the aorta.

At the beginning of ventricular diastole (i.e., ventricular filling) (see FIG. 2), the aortic and pulmonary valves are closed to prevent back flow from the arteries into the ventricles. Shortly thereafter, the tricuspid and mitral valves open (as FIG. 2 shows), to allow flow from the atriums into the corresponding ventricles. Shortly after ventricular systole (i.e., ventricular emptying) begins, the tricuspid and mitral valves close (see FIG. 3)—to prevent back flow from the ventricles into the corresponding atriums—and the aortic and pulmonary valves open—to permit discharge of blood into the arteries from the corresponding ventricles.

The opening and closing of heart valves occur primarily as a result of pressure differences. For example, the opening and closing of the mitral valve occurs as a result of the pressure differences between the left atrium and the left ventricle. During ventricular diastole, when ventricles are relaxed, the venous return of blood from the pulmonary veins into the left atrium causes the pressure in the atrium to exceed that in the ventricle. As a result, the mitral valve opens, allowing blood to enter the ventricle. As the ventricle contracts during ventricular systole, the intraventricular pressure rises above the pressure in the atrium and pushes the mitral valve shut.

The mitral and tricuspid valves are defined by fibrous rings of collagen, each called an annulus, which forms a part of the fibrous skeleton of the heart. The annulus provides attachments for the two cusps or leaflets of the mitral valve (called the anterior and posterior cusps) and the three cusps or leaflets of the tricuspid valve. The leaflets receive chordae tendineae from more than one papillary muscle. In a healthy heart, these muscles and their tendinous chords support the mitral and tricuspid valves, allowing the leaflets to resist the high pressure developed during contractions (pumping) of the left and right ventricles. FIGS. 5 and 6 show the chordae tendineae and papillary muscles in the left ventricle that support the mitral valve.

As FIGS. 2 and 3 show, the anterior (A) portion of the mitral valve annulus is intimate with the non-coronary leaflet of the aortic valve. As FIGS. 2 and 3 also show, the mitral valve annulus is also near other critical heart structures, such as the circumflex branch of the left coronary artery (which supplies the left atrium, a variable amount of the left ventricle, and in many people the SA node) and the AV node (which, with the SA node, coordinates the cardiac cycle).

Also in the vicinity of the posterior (P) mitral valve annulus is the coronary sinus and its tributaries. These vessels drain the areas of the heart supplied by the left coronary artery. The coronary sinus and its tributaries receive approximately 85% of coronary venous blood. The coronary sinus empties into the posterior of the right atrium, anterior and inferior to the fossa ovalis (see FIG. 4). A tributary of the coronary sinus is called the great cardiac vein, which courses parallel to the majority of the posterior mitral valve annulus, and is superior to the posterior mitral valve annulus by an average distance of about 9.64+/−3.15 millimeters (Yamanouchi, Y, Pacing and Clinical Electophysiology 21(11):2522-6; 1998).

### II. Characteristics and Causes of Mitral Valve Dysfunction

When the left ventricle contracts after filling with blood from the left atrium, the walls of the ventricle move inward and release some of the tension from the papillary muscle and chords. The blood pushed up against the under-surface of the mitral leaflets causes them to rise toward the annulus plane of the mitral valve. As they progress toward the annulus, the leading edges of the anterior and posterior leaflet come together forming a seal and closing the valve. In the healthy heart, leaflet coaptation occurs near the plane of the mitral annulus. The blood continues to be pressurized in the left ventricle until it is ejected into the aorta. Contraction of the papillary muscles is simultaneous with the contraction of the ventricle and serves to keep healthy valve leaflets tightly shut at peak contraction pressures exerted by the ventricle.

In a healthy heart (see FIGS. 7 and 8), the dimensions of the mitral valve annulus create an anatomic shape and tension such that the leaflets coapt, forming a tight junction, at peak contraction pressures. Where the leaflets coapt at the opposing medial (CM) and lateral (CL) sides of the annulus are called the leaflet commissures.

Valve malfunction can result from the chordae tendineae (the chords) becoming stretched, and in some cases tearing. When a chord tears, the result is a leaflet that flails. Also, a normally structured valve may not function properly because of an enlargement of or shape change in the valve annulus. This condition is referred to as a dilation of the annulus and generally results from heart muscle failure. In addition, the valve may be defective at birth or because of an acquired disease.

Regardless of the cause (see FIG. 9), mitral valve dysfunction can occur when the leaflets do not coapt at peak contraction pressures. As FIG. 9 shows, the coaptation line of the two leaflets is not tight at ventricular systole. As a result, an undesired back flow of blood from the left ventricle into the left atrium can occur.

Mitral regurgitation is a condition where, during contraction of the left ventricle, the mitral valve allows blood to flow backwards from the left ventricle into the left atrium. This has two important consequences.

First, blood flowing back into the atrium may cause high atrial pressure and reduce the flow of blood into the left atrium from the lungs. As blood backs up into the pulmonary system, fluid leaks into the lungs and causes pulmonary edema.

Second, the blood volume going to the atrium reduces volume of blood going forward into the aorta causing low cardiac output. Excess blood in the atrium over-fills the ventricle during each cardiac cycle and causes volume overload in the left ventricle.

Mitral regurgitation is measured on a numeric Grade scale of 1+ to 4+ by either contrast ventriculography or by echocardiographic Doppler assessment. Grade 1+ is trivial regurgitation and has little clinical significance. Grade 2+ shows a jet of reversed flow going halfway back into the left atrium. Grade 3 regurgitation shows filling of the left atrium with reversed flow up to the pulmonary veins and a contrast injection that clears in three heart beats or less. Grade 4 regurgitation has flow reversal into the pulmonary veins and a contrast injection that does not clear from the atrium in three or fewer heart beats.

Mitral regurgitation is categorized into two main types, (i) organic or structural and (ii) functional. Organic mitral regurgitation results from a structurally abnormal valve component that causes a valve leaflet to leak during systole. Functional mitral regurgitation results from annulus dilation due to primary congestive heart failure, which is itself generally surgically untreatable, and not due to a cause like severe irreversible ischemia or primary valvular heart disease.

Organic mitral regurgitation is seen when a disruption of the seal occurs at the free leading edge of the leaflet due to a ruptured chord or papillary muscle making the leaflet flail; or if the leaflet tissue is redundant, the valves may prolapse the level at which coaptation occurs higher into the atrium with further prolapse opening the valve higher in the atrium during ventricular systole.

Functional mitral regurgitation occurs as a result of dilation of heart and mitral annulus secondary to heart failure, most often as a result of coronary artery disease or idiopathic dilated cardiomyopathy. Comparing a healthy annulus in FIG. 7 to an unhealthy annulus in FIG. 9, the unhealthy annulus is dilated and, in particular, the anterior-to-posterior distance along the minor axis (line P-A) is increased. As a result, the shape and tension defined by the annulus becomes less oval (see FIG. 7) and more round (see FIG. 9). This condition is called dilation. When the annulus is dilated, the shape and tension conducive for coaptation at peak contraction pressures progressively deteriorate.

The fibrous mitral annulus is attached to the anterior mitral leaflet in one-third of its circumference. The muscular mitral annulus constitutes the remainder of the mitral annulus and is attached to by the posterior mitral leaflet. The anterior fibrous mitral annulus is intimate with the central fibrous body, the two ends of which are called the fibrous trigones. Just posterior to each fibrous trigone is the commissure of which there are two, the anterior medial (CM) and the posterior lateral commissure (CL). The commissure is where the anterior leaflet meets the posterior leaflet at the annulus.

As before described, the central fibrous body is also intimate with the non-coronary leaflet of the aortic valve. The central fibrous body is fairly resistant to elongation during the process of mitral annulus dilation. It has been shown that the great majority of mitral annulus dilation occurs in the posterior two-thirds of the annulus known as the muscular annulus. One could deduce thereby that, as the annulus dilates, the percentage that is attached to the anterior mitral leaflet diminishes.

In functional mitral regurgitation, the dilated annulus causes the leaflets to separate at their coaptation points in all phases of the cardiac cycle. Onset of mitral regurgitation may be acute, or gradual and chronic in either organic or in functional mitral regurgitation.

In dilated cardiomyopathy of ischemic or of idiopathic origin, the mitral annulus can dilate to the point of causing functional mitral regurgitation. It does so in approximately twenty-five percent of patients with congestive heart failure evaluated in the resting state. If subjected to exercise, echocardiography shows the incidence of functional mitral regurgitation in these patients rises to over fifty percent.

Functional mitral regurgitation is a significantly aggravating problem for the dilated heart, as is reflected in the increased mortality of these patients compared to otherwise comparable patients without functional mitral regurgitation. One mechanism by which functional mitral regurgitation aggravates the situation in these patients is through increased volume overload imposed upon the ventricle. Due directly to the leak, there is increased work the heart is required to perform in each cardiac cycle to eject blood antegrade through the aortic valve and retrograde through the mitral valve. The latter is referred to as the regurgitant fraction of left ventricular ejection. This is added to the forward ejection fraction to yield the total ejection fraction. A normal heart has a forward ejection fraction of about 50 to 70 percent. With functional mitral regurgitation and dilated cardiomyopathy, the total ejection fraction is typically less than thirty percent. If the regurgitant fraction is half the total ejection fraction in the latter group the forward ejection fraction can be as low as fifteen percent.

### III. Prior Treatment Modalities

In the treatment of mitral valve regurgitation, diuretics and/or vasodilators can be used to help reduce the amount of blood flowing back into the left atrium. An intra-aortic balloon counterpulsation device is used if the condition is not stabilized with medications. For chronic or acute mitral valve regurgitation, surgery to repair or replace the mitral valve is often necessary.

Currently, patient selection criteria for mitral valve surgery are very selective. Possible patient selection criteria for mitral surgery include: normal ventricular function, general good health, a predicted lifespan of greater than 3 to 5 years, NYHA Class III or IV symptoms, and at least Grade 3 regurgitation. Younger patients with less severe symptoms may be indicated for early surgery if mitral repair is anticipated. The most common surgical mitral repair procedure is for organic mitral regurgitation due to a ruptured chord on the middle scallop of the posterior leaflet.

In conventional annuloplasty ring repair, the posterior mitral annulus is reduced along its circumference with sutures passed through a surgical annuloplasty sewing ring cuff. The goal of such a repair is to bring the posterior mitral leaflet forward toward to the anterior leaflet to better allow coaptation.

Surgical edge-to-edge juncture repairs, which can be performed endovascularly, are also made, in which a mid-valve leaflet to mid-valve leaflet suture or clip is applied to keep these points of the leaflet held together throughout the cardiac cycle. Other efforts have developed an endovascular suture and a clip to grasp and bond the two mitral leaflets in the beating heart.

Grade 3+ or 4+ organic mitral regurgitation may be repaired with such edge-to-edge technologies. This is because, in organic mitral regurgitation, the problem is not the annulus but in the central valve components.

However, functional mitral regurgitation can persist at a high level, even after edge-to-edge repair, particularly in cases of high Grade 3+ and 4+ functional mitral regurgitation. After surgery, the repaired valve may progress to high rates of functional mitral regurgitation over time.

In yet another emerging technology, the coronary sinus is mechanically deformed through endovascular means applied and contained to function solely within the coronary sinus.

It is reported that twenty-five percent of the six million Americans who will have congestive heart failure will have functional mitral regurgitation to some degree. This constitutes the 1.5 million people with functional mitral regurgitation. Of these, the idiopathic dilated cardiomyopathy accounts for 600,000 people. Of the remaining 900,000 people with ischemic disease, approximately half have functional mitral regurgitation due solely to dilated annulus.

By interrupting the cycle of progressive functional mitral regurgitation, it has been shown in surgical patients that survival is increased and in fact forward ejection fraction increases in many patients. The problem with surgical therapy is the significant insult it imposes on these chronically ill patients with high morbidity and mortality rates associated with surgical repair.

The need remains for simple, cost-effective, and less invasive devices, systems, and methods for treating dysfunction of a heart valve, e.g., in the treatment of organic and functional mitral valve regurgitation.

### SUMMARY OF THE INVENTION

The invention comprises devices, systems, and methods for reshaping a heart valve annulus.

One aspect of the invention provides systems and methods comprising an implant adapted for spanning a heart valve, the implant adapted to extend through a first heart chamber and to a second heart chamber, and the implant adapted to generate a tension, the tension creating a force ranging between about 0.1 lbf to about 1.6 lbf.

An aspect of the invention provides systems and methods including steps of providing an implant adapted for spanning a heart valve, extending the implant through a first heart chamber and to second heart chamber, and applying a tension to the implant, the tension creating a force ranging between about 0.1 lbf to about 1.6 lbf.

Extending the implant may comprise extending the implant through a right atrium and to a left atrium, or extending the implant through a left atrium and to a right atrium. Extending the implant through the left atrium may include extending the implant through a posterior atrial wall, and/or extending the implant from a great cardiac vein and through a posterior atrial wall.

Extending the implant to the right atrium may include extending the implant through the septum, and/or extending the implant through the fossa ovalis.

The generated tension may result in an upward force and an inward force acting on a heart wall, where the heart wall may be a left ventricular wall. The generated tension may result in a downward force and an inward force acting on a septal wall, where the septal wall is a left atrial wall.

Additional steps may include measuring the tension on the implant, including providing a catheter including a flexural compliant member at a distal end, providing force sensing means coupled to a measurement device, positioning the force sensing means between the implant in at least one of the first heart chamber and the second heart chamber and the flexural compliant member, applying a tension to the implant while simultaneously pushing the catheter, and measuring the tension.

Another aspect of the invention provides systems and methods adapted for restoring coaptation of a heart valve. The systems and methods comprise an implant comprising a first angle, a second angle, and a force, and the first angle, the second angle, and the force being combined to produce a force projection having a range between about 0.04 lbf to about 1.58 lbf. The first angle may comprise a vertex positioned at or near a great cardiac vein, with a range between about 10 degrees and about 60 degrees. The second angle may comprise a vertex positioned at or near a great cardiac vein, with a range between about zero degrees and about 45 degrees. The force may comprise a range between about 0.1 lbf to about 1.6 lbf.

The force generated may comprise an upward force and an inward force acting on a heart wall, and the heart wall may be a left ventricular wall. The force may also comprise a downward force and an inward force acting on a septal wall, and the septal wall may be a left atrial wall.

Yet another aspect of the invention provides systems and methods including steps of providing an implant, extending the implant through a first heart chamber and to a second heart chamber, the implant comprising a first angle, a second angle, and a force, the first angle, the second angle, and the force combining to produce a force projection having a range from about 0.04 lbf to about 1.58 lbf, and restoring coaptation of the heart valve. Extending the implant may comprise extending the implant through a left atrium and to a right atrium. The force may comprise a range between about 0.1 lbf to about 1.6 lbf.

Another aspect of the invention provides systems and methods of measuring the tension on a heart implant, with steps including providing a catheter including a flexural compliant member at a distal end, providing force sensing means coupled to a measurement device, positioning the force sensing means between the implant and the flexural compliant member, applying a tension to the implant while simultaneously pushing the catheter, and measuring the tension. The force sensing means may be positioned between the implant in a heart chamber and the flexural compliant member. The heart chamber may comprise a left atrium or a right atrium or a left ventricle or a right ventricle.

The tension generated may comprise an upward tension and an inward tension acting on a heart wall, and the heart wall may be a left ventricular wall. The tension may also comprise a downward tension and an inward tension acting on a septal wall, and the septal wall may be a left atrial wall.

Another aspect of the invention provides systems and methods of measuring the tension on a heart implant, the systems and methods comprising an implant adapted to extend through a first heart chamber and to a second heart chamber, a catheter including a flexural compliant member at a distal end, force sensing means coupled to a measurement device, the force sensing means positioned between the implant and the flexural compliant member, with the implant adapted to generate a tension while the catheter is simultaneously pushed, and the measurement device adapted to provide a measurement of the tension.

Other features and advantages of the invention shall be apparent based upon the accompanying description, drawings, and claims.

### BRIEF DESCRIPTION OF THE DRAWINGS

FIG. 1 is an anatomic anterior view of a human heart, with portions broken away and in section to view the interior heart chambers and adjacent structures.

FIG. 2 is an anatomic superior view of a section of the human heart showing the tricuspid valve in the right atrium, the mitral valve in the left atrium, and the aortic valve in between, with the tricuspid and mitral valves open and the aortic and pulmonary valves closed during ventricular diastole (ventricular filling) of the cardiac cycle.

FIG. 3 is an anatomic superior view of a section of the human heart shown in FIG. 2, with the tricuspid and mitral valves closed and the aortic and pulmonary valves opened during ventricular systole (ventricular emptying) of the cardiac cycle.

FIG. 4 is an anatomic anterior perspective view of the left and right atriums, with portions broken away and in section to show the interior of the heart chambers and associated structures, such as the fossa ovalis, coronary sinus, and the great cardiac vein.

FIG. 5 is an anatomic lateral view of a human heart with portions broken away and in section to show the interior of the left ventricle and associated muscle and chord structures coupled to the mitral valve.

FIG. 6 is an anatomic lateral view of a human heart with portions broken away and in section to show the interior of the left ventricle and left atrium and associated muscle and chord structures coupled to the mitral valve.

FIG. 7 is a superior view of a healthy mitral valve, with the leaflets closed and coapting at peak contraction pressures during ventricular systole.

FIG. 8 is an anatomic superior view of a section of the human heart, with the normal mitral valve shown in FIG. 7 closed during ventricular systole (ventricular emptying) of the cardiac cycle.

FIG. 9 is a superior view of a dysfunctional mitral valve, with the leaflets failing to coapt during peak contraction pressures during ventricular systole, leading to mitral regurgitation.

FIGS. 10A and 10B are anatomic anterior perspective views of the left and right atriums, with portions broken away and in section to show the presence of an implant system that includes an inter-atrial bridging element that spans the mitral valve annulus, with a posterior bridge stop positioned in the great cardiac vein and an anterior bridge stop, including a septal member, positioned on the inter-atrial septum, the inter-atrial bridging element extending in an essentially straight path generally from a mid-region of the annulus to the inter-atrial septum.

FIG. 10C is an anatomic anterior perspective view of an alternative embodiment of the implant system shown in FIGS. 10A and 10B, showing an anterior bridge stop without the addition of a septal member.

FIG. 11 is an anatomic anterior perspective view of the left and right atriums, with portions broken away and in section to show the presence of an implant system of the type shown in FIGS. 10A to 10C, with the anterior region of the implant situated on the inter-atrial septum, as well as in the superior vena cava and the inferior vena cava.

FIG. 12 is an anatomic anterior perspective view of the left and right atriums, with portions broken away and in section to show the presence of an implant system that includes an inter-atrial bridging element that spans the mitral valve annulus, with a posterior region situated in the great cardiac vein and an anterior region situated on the interatrial septum, the inter-atrial bridging element extending in a curvilinear path, bending around a trigone of the annulus generally from a mid-region region of the annulus, as well as elevating in an arch toward the dome of the left atrium.

FIG. 13 is an anatomic anterior perspective view of the left and right atriums, with portions broken away and in section to show the presence of an implant system that includes three inter-atrial bridging elements that span the mitral valve annulus, each with a posterior region situated in the great cardiac vein and an anterior region situated on the interatrial septum, two of the inter-atrial bridging elements extending in generally straight paths from different regions of the annulus, and the third inter-atrial bridging elements extending in a generally curvilinear path toward a trigone of the annulus.

FIG. 14A is a side view of a septal member which may be used as part of the implant system of the type shown in FIGS. 10A and 10B.

FIG. 14B is a side view of a deployed septal member of the type shown in FIG. 14A, showing the member sandwiching portions of the septum through an existing hole.

FIGS. 15A and 15B are sectional views showing the ability of a bridge stop used in conjunction with the implant shown in FIGS. 10A to 10C to move back and forth independent of the septal wall and inner wall of the great cardiac vein.

FIG. 16 is an anatomic perspective view showing the use of a GCV catheter and an LA catheter configured for establishing the posterior bridge stop region.

FIG. 17 is an anatomic perspective view showing the use of a GCV catheter and an LA catheter configured for establishing the trans-septal bridging element.

FIG. 18 is an anatomic perspective view showing the use of an LA catheter configured for establishing the anterior bridge stop region.

FIG. 19 is a close-up perspective view showing the use of a bridge stop attached to the bridging element and abutting the septal member.

FIG. 20 is an anatomic section view of the left atrium and associated mitral valve structure, showing mitral dysfunction.

FIG. 21 is an anatomic superior view of a section of the human heart, showing the presence of an implant system of the type shown in FIGS. 10A and 10B, and showing proper coaptation of the mitral valve leaflets.

FIG. 22 is an anatomic section view of the implant system taken generally along line 22-22 in FIG. 21, showing the presence of an implant system of the type shown in FIGS. 10A and 10B, and showing proper coaptation of the mitral valve leaflets.

FIG. 23 is an anatomic partial view of a patient depicting access points used for implantation of an implant system, and also showing a loop guide wire accessible to the exterior the body at two locations.

FIG. 24 is an anatomic view depicting a representative alternative catheter-based device for implanting an implant system of the type shown in FIGS. 10A to 10C, and showing a bridging element being pulled through the vasculature structure by a loop guide wire.

FIG. 25 is an anatomic partial view of a patient showing a bridge stop connected to a bridging element in preparation to be pulled and/or pushed through the vasculature structure and positioned within the great cardiac vein.

FIG. 26 is an anatomic view depicting a representative alternative catheter-based device for implanting a system of the type shown in FIGS. 10A to 10C, and showing a bridge stop being positioned within the great cardiac vein.

FIGS. 27 and 28 are anatomic partial views of a patient depicting access points used for implantation of an implant system, and also showing a loop guide wire accessible to the exterior the body at a single location (femoral vein).

FIG. 29 is a perspective view of a symmetrically shaped T-shaped bridge stop or member which may be used with the implant system of the type shown in FIGS. 10A to 10C.

FIG. 30 is a perspective view of an alternative embodiment of the T-shaped bridge stop shown in FIG. 29, showing the bridge stop being asymmetric and having one limb shorter than the other.

FIGS. 31A to 31G are perspective and sectional views of an alternative embodiment of a bridge stop and associated delivery tool.

FIGS. 32A to 32C are perspective views of the alternative embodiment of the bridge stop shown in FIGS. 31A to 31G, and showing the use of the delivery tool.

FIG. 33 is a diagrammatic superior view of the implant system shown in FIGS. 10A to 10C, and showing the minor axis force projection MAFP (force vector), the lateral angle theta, the vertical angle phi, and the bridge force BF (force vector) applied to the bridging element.

FIG. 34 is a diagrammatic lateral view of the implant system shown in FIGS. 10A to 10C, similar to FIG. 33, and showing the minor axis force projection MAFP (force vector), the lateral angle theta, the vertical angle phi, and the bridge force BF (force vector) applied to the bridging element.

FIG. 35 is a perspective view of a load cell or similar force sensing device adapted to provide a measure of the bridge force BF magnitude applied to the bridging element of the implant system 10.

FIG. 36 is a perspective view of the load cell shown in FIG. 35, the load cell positioned between a septal member and a catheter to provide a magnitude of the bridge force BF.

FIG. 37 is a table showing representative ranges of the lateral angle theta, the superior angle phi, and bridge force BF magnitudes, that when combined produce a minor axis force projection MAFP, with minimum, typical, and maximum magnitude values shown.

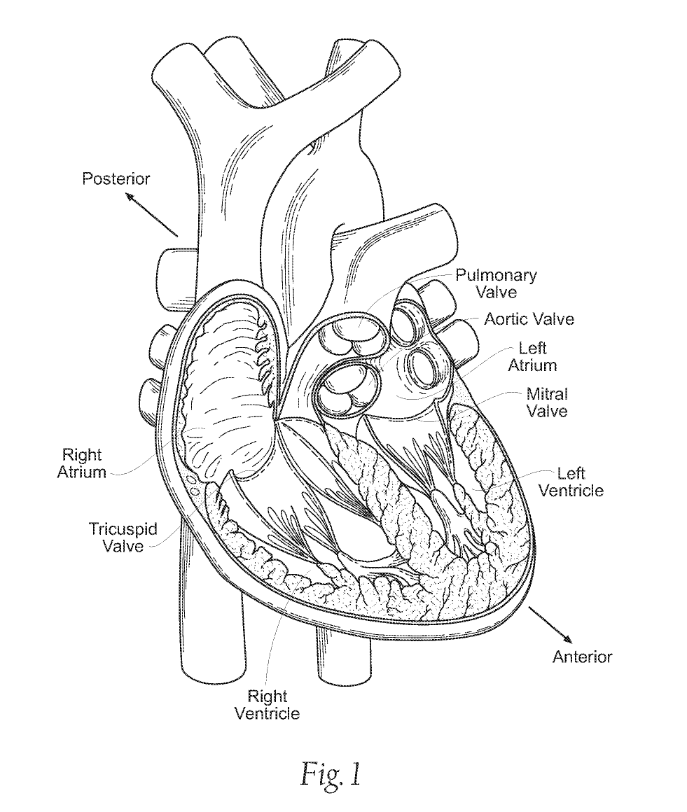

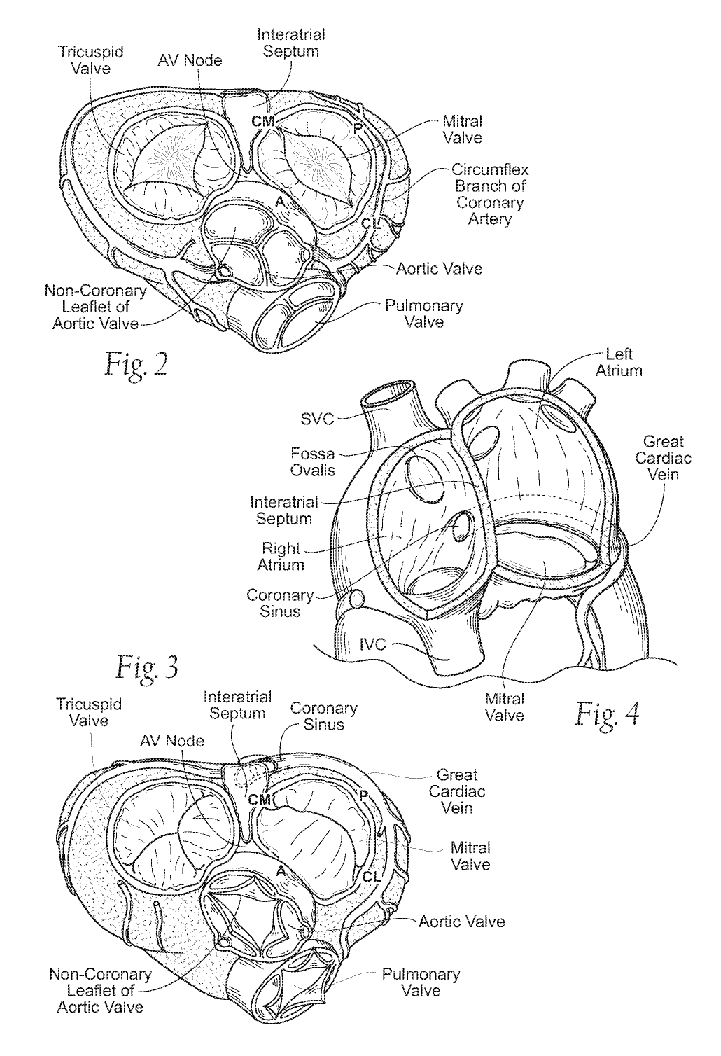

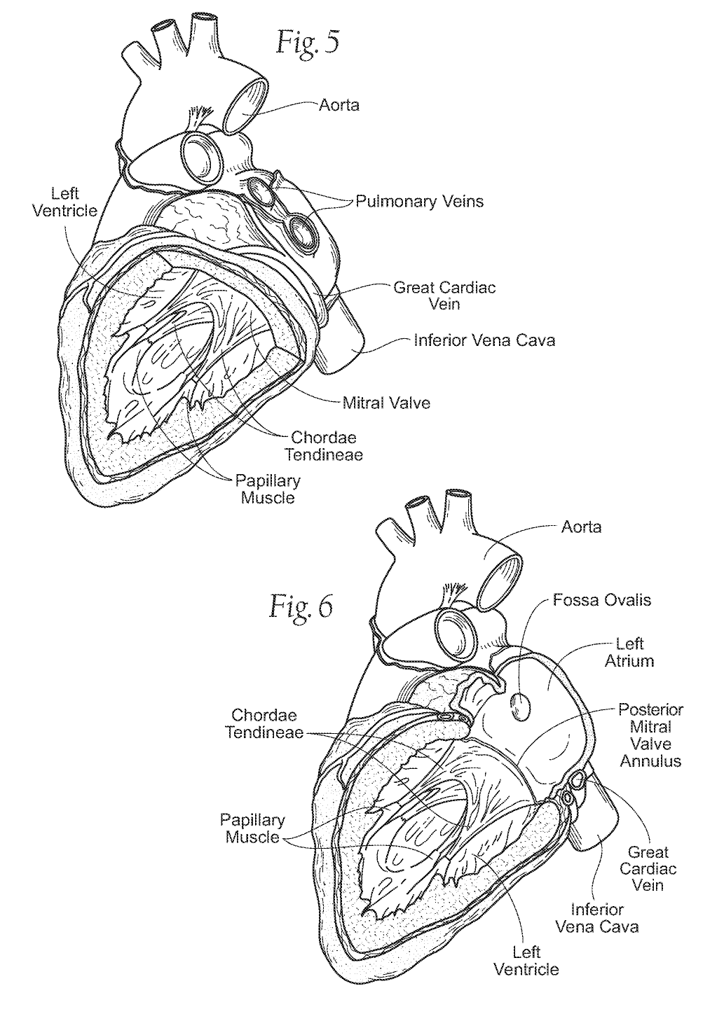

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

### I. Trans-Septal Implants for Direct Shortening of the Minor Axis of a Heart Valve Annulus

A. Implant Structure

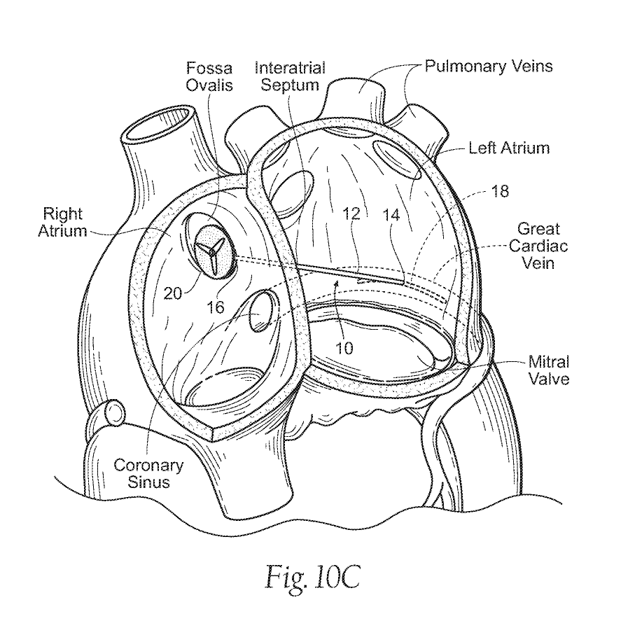

FIGS. 10A to 10C show embodiments of an implant 10 that is sized and configured to extend across the left atrium in generally an anterior-to-posterior direction, spanning the mitral valve annulus. The implant 10 comprises a spanning region or bridging element 12 having a posterior bridge stop region 14 and an anterior bridge stop region 16.

The posterior bridge stop region 14 is sized and configured to allow the bridging element 12 to be placed in a region of atrial tissue above the posterior mitral valve annulus. This region is preferred, because it generally presents more tissue mass for obtaining purchase of the posterior bridge stop region 14 than in a tissue region at or adjacent to the posterior mitral annulus. Engagement of tissue at this supra-annular location also may reduce risk of injury to the circumflex coronary artery.

In a small percentage of cases, the circumflex coronary artery may pass over and medial to the great cardiac vein on the left atrial aspect of the great cardiac vein, coming to lie between the great cardiac vein and endocardium of the left atrium. However, since the forces in the posterior bridge stop region are directed upward and inward relative to the left atrium and not in a constricting manner along the long axis of the great cardiac vein, the likelihood of circumflex artery compression is less compared to other technologies in this field that do constrict the tissue of the great cardiac vein. Nevertheless, should a coronary angiography reveal circumflex artery stenosis, the symmetrically shaped posterior bridge stop may be replaced by an asymmetrically shaped bridge stop, such as where one limb of a T-shaped member is shorter than the other, thus avoiding compression of the crossing point of the circumflex artery. The asymmetric form may also be selected first based on a pre-placement angiogram.

An asymmetric posterior bridge stop may be utilized for other reasons as well. The asymmetric posterior bridge stop may be selected where a patient is found to have a severely stenotic distal great cardiac vein, where the asymmetric bridge stop better serves to avoid obstruction of that vessel. In addition, an asymmetric bridge stop may be chosen for its use in selecting application of forces differentially and preferentially on different points along the posterior mitral annulus to optimize treatment, i.e., in cases of malformed or asymmetrical mitral valves.

The anterior bridge stop region 16 is sized and configured to allow the bridging element 12 to be placed, upon passing into the right atrium through the septum, adjacent tissue in or near the right atrium. For example, as is shown in FIGS. 10A to 10C, the anterior bridge stop region 16 may be adjacent or abutting a region of fibrous tissue in the interatrial septum. As shown, the bridge stop site 16 is desirably superior to the anterior mitral annulus at about the same elevation or higher than the elevation of the posterior bridge stop region 14.

In the illustrated embodiment, the anterior bridge stop region 16 is adjacent to or near the inferior rim of the fossa ovalis. Alternatively, the anterior bridge stop region 16 can be located at a more superior position in the septum, e.g., at or near the superior rim of the fossa ovalis. The anterior bridge stop region 16 can also be located in a more superior or inferior position in the septum, away from the fossa ovalis, provided that the bridge stop site does not harm the tissue region.

Alternatively, as can be seen in FIG. 11, the anterior bridge stop region 16, upon passing through the septum into the right atrium, may be positioned within or otherwise situated in the superior vena cava (SVC), the inferior vena cava (IVC), or a combination of both, instead of at the septum itself.

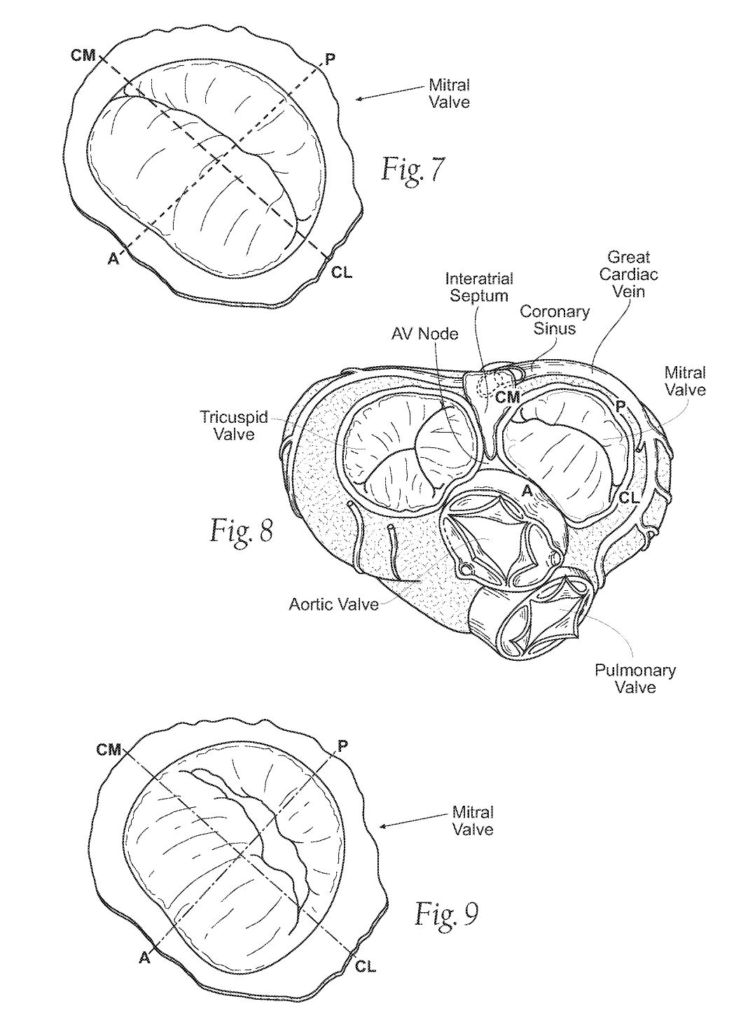

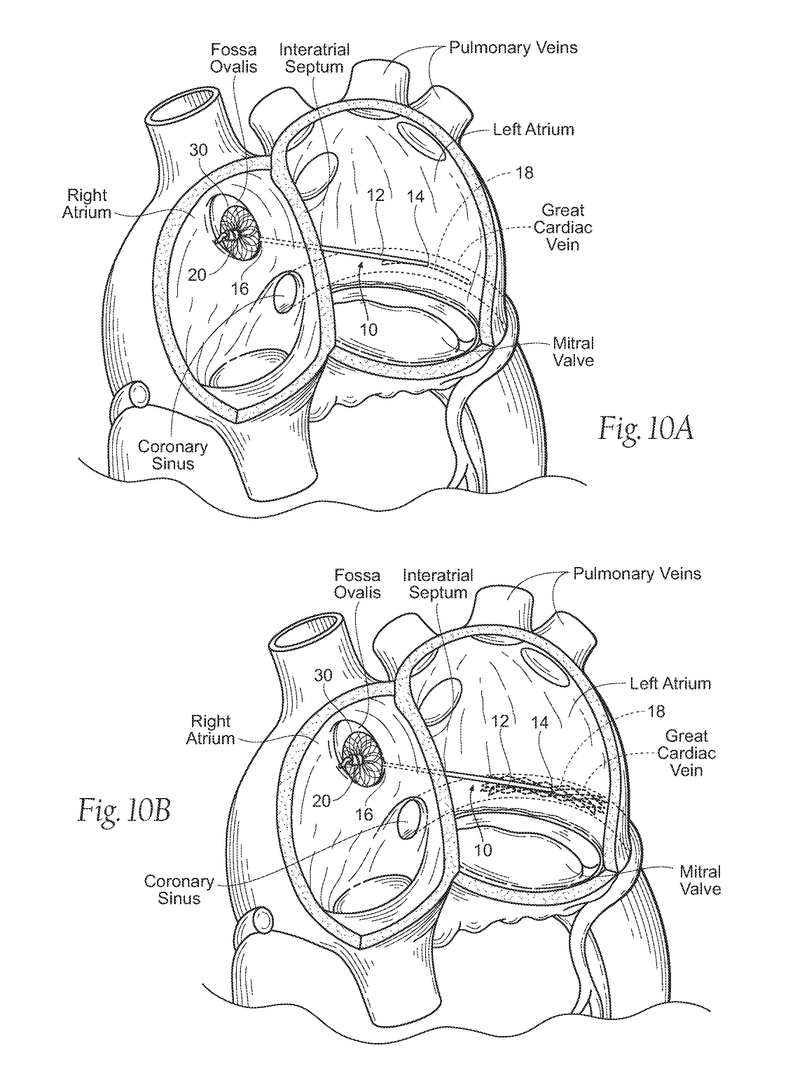

In use, the spanning region or bridging element 12 can be placed into tension between the two bridge stop regions 14 and 16. The implant 10 thereby serves to apply a direct mechanical force generally in a posterior to anterior direction across the left atrium. The direct mechanical force can serve to shorten the minor axis (line P-A in FIG. 7) of the annulus. In doing so, the implant 10 can also reactively reshape the annulus along its major axis (line CM-CL in FIG. 7) and/or reactively reshape other surrounding anatomic structures. It should be appreciated, however, the presence of the implant 10 can serve to stabilize tissue adjacent the heart valve annulus, without affecting the length of the minor or major axes.

It should also be appreciated that, when situated in other valve structures, the axes affected may not be the “major” and “minor” axes, due to the surrounding anatomy. In addition, in order to be therapeutic, the implant 10 may only need to reshape the annulus during a portion of the heart cycle, such as during late diastole and early systole when the heart is most full of blood at the onset of ventricular systolic contraction, when most of the mitral valve leakage occurs. For example, the implant 10 may be sized to restrict outward displacement of the annulus during late ventricular diastolic relaxation as the annulus dilates.

The mechanical force applied by the implant 10 across the left atrium can restore to the heart valve annulus and leaflets a more normal anatomic shape and tension. The more normal anatomic shape and tension are conducive to coaptation of the leaflets during late ventricular diastole and early ventricular systole, which, in turn, reduces mitral regurgitation.

In its most basic form, the implant 10 is made from a biocompatible metallic or polymer material, or a metallic or polymer material that is suitably coated, impregnated, or otherwise treated with a material to impart biocompatibility, or a combination of such materials. The material is also desirably radio-opaque or incorporates radio-opaque features to facilitate fluoroscopic visualization.

The implant 10 can be formed by bending, shaping, joining, machining, molding, or extrusion of a metallic or polymer wire form structure, which can have flexible or rigid, or inelastic or elastic mechanical properties, or combinations thereof. Alternatively, the implant 10 can be formed from metallic or polymer thread-like or suture material. Materials from which the implant 10 can be formed include, but are not limited to, stainless steel, Nitinol, titanium, silicone, plated metals, Elgiloy™, NP55, and NP57.

The implant 10 can take various shapes and have various cross-sectional geometries. The implant 10 can have, e.g., a generally curvilinear (i.e., round or oval) cross-section, or a generally rectilinear cross section (i.e., square or rectangular), or combinations thereof. Shapes that promote laminar flow and therefore reduce hemolysis are contemplated, with features such as smoother surfaces and longer and narrower leading and trailing edges in the direction of blood flow.

B. The Posterior Bridge Stop Region

The posterior bridge stop region 14 is sized and configured to be located within or at the left atrium at a supra-annular position, i.e., positioned within or near the left atrium wall above the posterior mitral annulus.

In the illustrated embodiment, the posterior bridge stop region 14 is shown to be located generally at the level of the great cardiac vein, which travels adjacent to and parallel to the majority of the posterior mitral valve annulus. This tributary of the coronary sinus can provide a strong and reliable fluoroscopic landmark when a radio-opaque device is placed within it or contrast dye is injected into it. As previously described, securing the bridging element 12 at this supra-annular location also lessens the risk of encroachment of and risk of injury to the circumflex coronary artery compared to procedures applied to the mitral annulus directly. Furthermore, the supra-annular position assures no contact with the valve leaflets therefore allowing for coaptation and reduces the risk of mechanical damage.

The great cardiac vein also provides a site where relatively thin, non-fibrous atrial tissue can be readily augmented and consolidated. To enhance hold or purchase of the posterior bridge stop region 14 in what is essentially non-fibrous heart tissue, and to improve distribution of the forces applied by the implant 10, the posterior bridge stop region 14 may include a posterior bridge stop 18 placed within the great cardiac vein and abutting venous tissue. This makes possible the securing of the posterior bridge stop region 14 in a non-fibrous portion of the heart in a manner that can nevertheless sustain appreciable hold or purchase on that tissue for a substantial period of time, without dehiscence, expressed in a clinically relevant timeframe.

C. The Anterior Bridge Stop Region

The anterior bridge stop region 16 is sized and configured to allow the bridging element 12 to remain firmly in position adjacent or near the fibrous tissue and the surrounding tissues in the right atrium side of the atrial septum. The fibrous tissue in this region provides superior mechanical strength and integrity compared with muscle and can better resist a device pulling through. The septum is the most fibrous tissue structure in its own extent in the heart. Surgically handled, it is usually one of the only heart tissues into which sutures actually can be placed and can be expected to hold without pledgets or deep grasps into muscle tissue, where the latter are required.

As FIGS. 10A to 10C show, the anterior bridge stop region 16 passes through the septal wall at a supra-annular location above the plane of the anterior mitral valve annulus. The supra-annular distance on the anterior side can be generally at or above the supra-annular distance on the posterior side. As before pointed out, the anterior bridge stop region 16 is shown in FIGS. 10A to 10C at or near the inferior rim of the fossa ovalis, although other more inferior or more superior sites can be used within or outside the fossa ovalis, taking into account the need to prevent harm to the septal tissue and surrounding structures.

By locating the bridging element 12 at this supra-annular level within the right atrium, which is fully outside the left atrium and spaced well above the anterior mitral annulus, the implant 10 avoids the impracticalities of endovascular attachment at or adjacent to the anterior mitral annulus, where there is just a very thin rim of annulus tissue that is bounded anteriorly by the anterior leaflet, inferiorly by the aortic outflow tract, and medially by the atrioventricular (AV) node of the conduction system. The anterior mitral annulus is where the non-coronary leaflet of the aortic valve attaches to the mitral annulus through the central fibrous body. Anterior location of the implant 10 in the supra-annular level within the right atrium (either in the septum or in a vena cava) avoids encroachment of and risk of injury to both the aortic valve and the AV node.

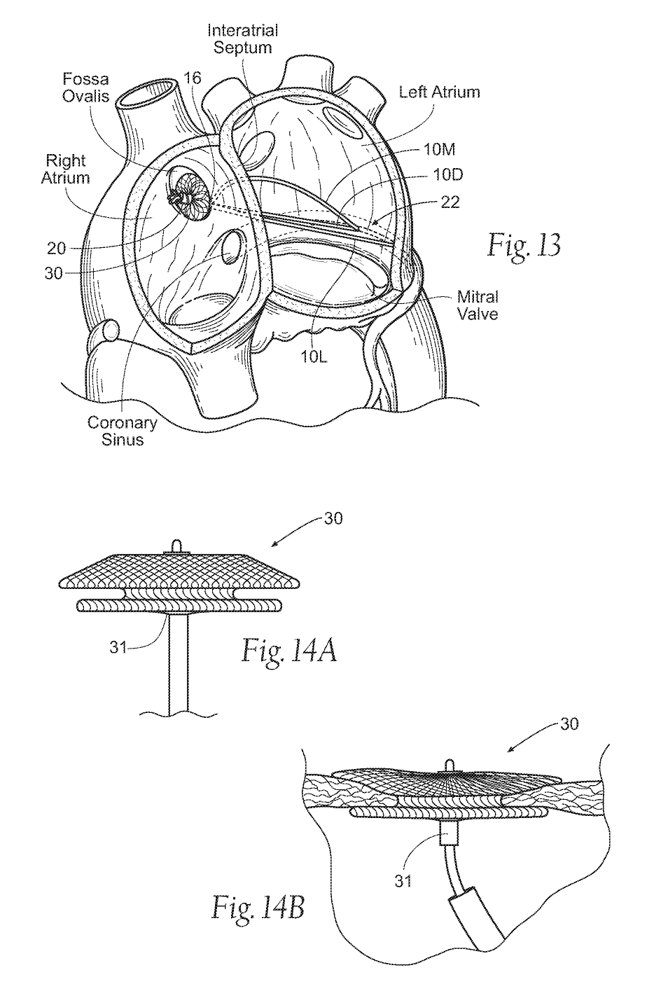

The purchase of the anterior bridge stop region 16 in fibrous septal tissue is desirably enhanced by a septal member 30 or an anterior bridge stop 20, or a combination of both. FIGS. 10A and 10B show the anterior bridge stop region including a septal member 30. FIG. 10C shows the anterior bridge stop region without a septal member. The septal member 30 may be an expandable device and also may be a commercially available device such as a septal occluder, e.g., Amplatzer® PEO Occluder (see FIGS. 14A and 14B). The septal member 30 preferably mechanically amplifies the hold or purchase of the anterior bridge stop region 16 in the fibrous tissue site. The septal member 30 also desirably increases reliance, at least partly, on neighboring anatomic structures of the septum to make firm the position of the implant 10. In addition, the septal member 30 may also serve to plug or occlude the small aperture that was created in the fossa ovalis or surrounding area during the implantation procedure.

Anticipating that pinpoint pulling forces will be applied by the anterior bridge stop region 16 to the septum, the forces acting on the septal member 30 should be spread over a moderate area, without causing impingement on valve, vessels or conduction tissues. With the pulling or tensioning forces being transmitted down to the annulus, shortening of the minor axis is achieved. A flexurally stiff septal member is preferred because it will tend to cause less focal narrowing in the direction of bridge element tension of the left atrium as tension on the bridging element is increased. The septal member 30 should also have a low profile configuration and highly washable surfaces to diminish thrombus formation for devices deployed inside the heart.

The septal member may also have a collapsed configuration and a deployed configuration. The septal member 30 may also include a hub 31 (see FIGS. 14A and 14B) to allow attachment of the bridge stop 20. A septal brace may also be used in combination with the septal member 30 and anterior bridge stop 20 to distribute forces uniformly along the septum (see FIG. 11). Alternatively, devices in the IVC or the SVC can be used as bridge stop sites, instead of confined to the septum.

Location of the posterior and anterior bridge stop regions 14 and 16 having radio-opaque bridge locks and well demarcated fluoroscopic landmarks respectively at the supra-annular tissue sites just described, not only provides freedom from key vital structure damage or local impingement—e.g., to the circumflex artery, AV node, and the left coronary and non-coronary cusps of the aortic valve—but the supra-annular focused sites are also not reliant on purchase between tissue and direct tension-loaded penetrating/biting/holding tissue attachment mechanisms. Instead, physical structures and force distribution mechanisms such as stents, T-shaped members, and septal members can be used, which better accommodate the attachment or abutment of mechanical levers and bridge locks, and through which potential tissue tearing forces can be better distributed.

Further, the bridge stop sites 14, 16 do not require the operator to use complex imaging. Adjustment of implant position after or during implantation is also facilitated, free of these constraints. The bridge stop sites 14, 16 also make possible full intra-atrial retrieval of the implant 10 by endovascularly snaring and then cutting the bridging element 12 at either side of the left atrial wall, from which it emerges.

D. Orientation of the Bridging Element

In the embodiments shown in FIGS. 10A to 10C, the implant 10 is shown to span the left atrium beginning at a posterior point of focus superior to the approximate mid-point of the mitral valve annulus, e.g., at the P2 posterior mitral leaflet, and proceeding in an anterior direction in a generally straight path directly to the region of anterior focus in the septum. As shown in FIGS. 10A to 10C, the spanning region or bridging element 12 of the implant 10 may be preformed or otherwise configured to extend in this essentially straight path above the plane of the valve, without significant deviation in elevation toward or away from the plane of the annulus, other than as dictated by any difference in elevation between the posterior and anterior regions of placement.

Lateral or medial deviations and/or superior or inferior deviations in this path can be imparted, if desired, to affect the nature and direction of the force projection (e.g., force vector), that the implant 10 applies (to be discussed in greater detail in Section III). It should be appreciated that the spanning region or bridging element 12 can be preformed or otherwise configured with various medial/lateral and/or inferior/superior deviations to achieve targeted annulus and/or atrial structure remodeling, which takes into account the particular therapeutic needs and morphology of the patient. In addition, deviations in the path of the bridging element may also be imparted in order to avoid the high velocity blood path within a heart chamber, such as the left atrium.

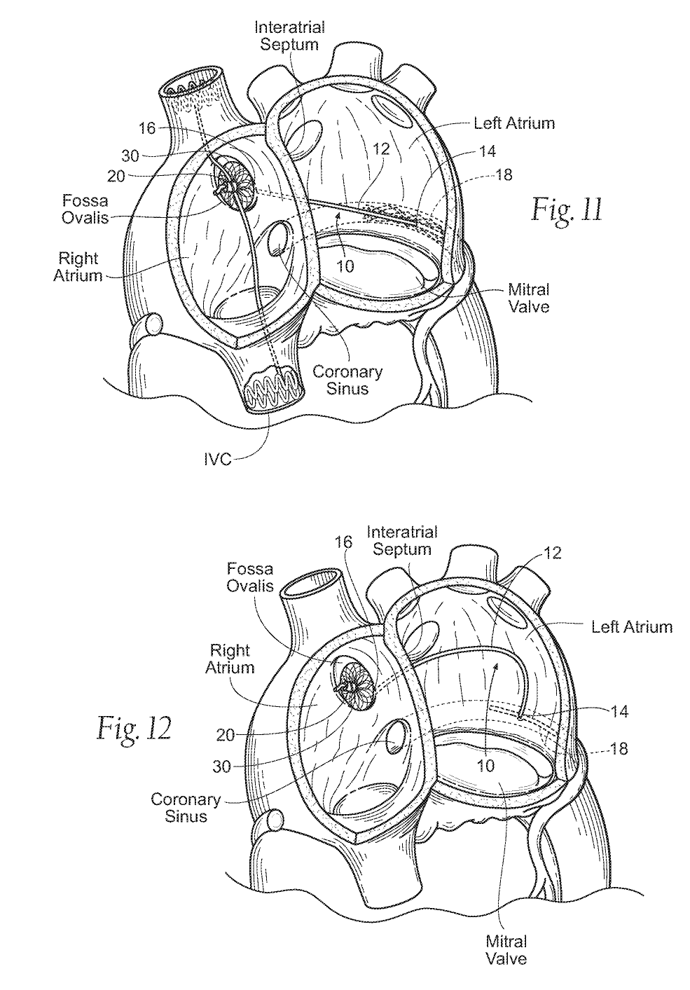

As non-limiting examples, FIGS. 12 and 13 show alternative configurations of the implant 10. For example, as shown in FIG. 12, the spanning region or bridging element 12 can follow a curvilinear path bending around a trigone (medial or lateral) of the annulus as well as elevate in an arch away from the plane of the valve.

FIG. 13 shows a system 22 comprising a direct middle implant 10D, a medial curvilinear implant 10M, and a direct lateral implant 10L. One, two, or all of the implants 10 can be parallel to the valve, or arch upward, or bend downward, as previously described.

E. Posterior and Anterior Bridge Stop

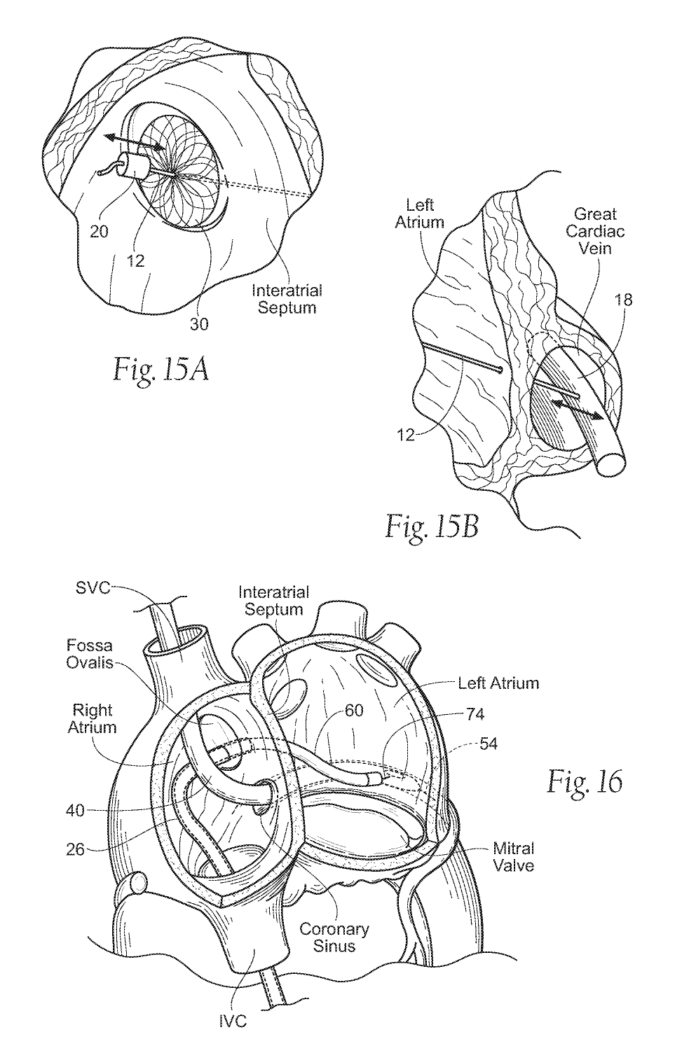

It is to be appreciated that a bridge stop as described herein, including a posterior or anterior bridge stop, describes an apparatus that may releasably hold the bridging element 12 in a tensioned state. As can be seen in FIGS. 15A and 15B, bridge stops 20 and 18 respectively are shown releasably secured to the bridging element 12, allowing the bridge stop structure to move back and forth independent of the inter-atrial septum and inner wall of the great cardiac vein during a portion of the cardiac cycle when the tension force may be reduced or becomes zero. Alternative embodiments are also possible, all of which may provide this function. It is also to be appreciated that the general descriptions of posterior and anterior are non-limiting to the bridge stop function, i.e., a posterior bridge stop may be used anterior, and an anterior bridge stop may be used posterior.

When the bridge stop is in an abutting relationship to a septal member or a T-shaped member, for example, the bridge stop allows the bridging element to move freely within or around the septal member or T-shaped member, i.e., the bridging element is not connected to the septal member or T-shaped member. In this configuration, the bridging element is held in tension by the bridge stop, whereby the septal member or T-shaped member serves to distribute the force applied by the bridging element across a larger surface area. Alternatively, the bridge stop may be mechanically connected to the septal member or T-shaped member, e.g., when the bridge stop is positioned over and secured to the septal member hub. In this configuration, the bridging element is fixed relative to the septal member position and is not free to move about the septal member.

### II. General Methods of Trans-Septal Implantation

The implants 10 or implant systems 22 as just described lend themselves to implantation in a heart valve annulus in various ways. The implants 10 or implant systems 22 can be implanted, e.g., in an open heart surgical procedure. Alternatively, the implants 10 or implant systems 22 can be implanted using catheter-based technology via a peripheral venous access site, such as in the femoral or jugular vein (via the IVC or SVC) under image guidance, or trans-arterial retrograde approaches to the left atrium through the aorta from the femoral artery also under image guidance.

Alternatively, the implants 10 or implant systems 22 can be implanted using thoracoscopic means through the chest, or by means of other surgical access through the right atrium, also under image guidance. Image guidance includes but is not limited to fluoroscopy, ultrasound, magnetic resonance, computed tomography, or combinations thereof.

The implants 10 or implant systems 22 may comprise independent components that are assembled within the body to form an implant, or alternatively, independent components that are assembled exterior the body and implanted as a whole.

Percutaneous vascular access may be achieved by conventional methods into the femoral or jugular vein, or a combination of both. Under image guidance, a first catheter, or great cardiac vein catheter 40, and a second catheter, or left atrium catheter 60, are steered through the vasculature into the right atrium. It is a function of the great cardiac vein (GCV) catheter 40 and left atrium (LA) catheter 60 to establish the posterior bridge end stop region. Catheter access to the right and left atriums can be achieved through either a femoral vein to IVC or SVC route (in the latter case, for a caval brace) or an upper extremity or neck vein to SVC or IVC route (in the latter case, for a caval brace). In the case of the SVC, the easiest access is from the upper extremity or neck venous system; however, the IVC can also be accessed by passing through the SVC and right atrium. Similarly the easiest access to the IVC is through the femoral vein; however the SVC can also be accessed by passing through the IVC and right atrium.

A first implantation step can be generally described as establishing the posterior bridge stop region 14. As can be seen in FIG. 16, the GCV catheter 40 is steered through the vasculature into the right atrium. The GCV catheter 40 is then steered through the coronary sinus and into the great cardiac vein. The second catheter, or LA catheter 60, is also steered through the vasculature and into the right atrium. The LA catheter 60 then passes through the septal wall at or near the fossa ovalis and enters the left atrium. A catheter 26 may be provided to assist the guidance of the LA catheter 60 into the left atrium. Once the GCV catheter 40 and the LA catheter 60 are in their respective positions in the great cardiac vein and left atrium, it is a function of the GCV and LA catheters 40, 60 to configure the posterior bridge stop region 14.

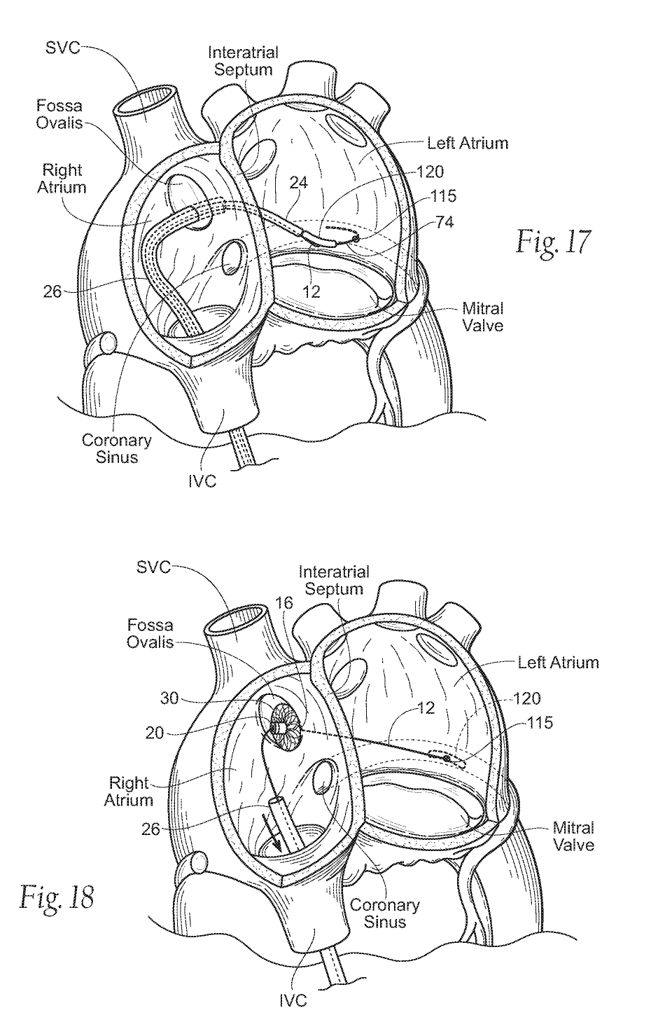

A second step can be generally described as establishing the trans-septal bridging element 12. A deployment catheter 24 via the LA catheter 60 is used to position a posterior bridge stop 18 and a preferably preattached and predetermined length of bridging element 12 within the great cardiac vein (see FIG. 17). The predetermined length of bridging element 12, e.g., two meters, extends from the posterior bridge stop 18, through the left atrium, through the fossa ovalis, through the vasculature, and preferably remains accessible exterior the body. The predetermined length of bridging element may be cut or detached in a future step, leaving implanted the portion extending from the posterior bridge stop 18 to the anterior bridge stop 20. Alternatively, the bridging element 12 may not be cut or detached at the anterior bridge stop 20, but instead the bridging element 12 may be allowed to extend into the IVC for possible future retrieval.

A third step can be generally described as establishing the anterior bridge stop region 16 (see FIGS. 18 and 19). The bridging element 12 is first threaded through the septal member 30. The septal member 30 is then advanced over the bridging element 12 in a collapsed condition through catheter 26, and is positioned and deployed at or near the fossa ovalis within the right atrium. A bridge stop 20 may be attached to the bridging element 12 and advanced with the septal member 30, or alternatively, the bridge stop 20 may be advanced to the right atrium side of the septal member 30 after the septal member has been positioned or deployed.

A fourth step can be generally described as adjusting the bridging element 12 for proper therapeutic effects. With the posterior bridge stop region 14, bridging element 12, and anterior bridge stop region 16 configured as previously described, a tension is placed on the bridging element 12. The implant 10 and associated regions may be allowed to settle for a predetermined amount of time, e.g., five or more seconds. The mitral valve and mitral valve regurgitation are observed for desired therapeutic effects. The tension on the bridging element 12 may be adjusted until a desired result is achieved. The bridge stop 20 is then allowed to secure the bridging element 12 when the desired tension or measured length or degree of mitral regurgitation reduction is achieved.

Further details of representative embodiments of the deployment of an implant 10 of the types shown in FIGS. 10A to 10C by a percutaneous, catheter-based procedure, under image guidance can be found in co-pending, commonly owned U.S. patent application Ser. No. 11/389,819, filed Mar. 27, 2006, and entitled “Devices, Systems, and Methods for Reshaping a Heart Valve Annulus,” which is incorporated herein by reference.

A. Implantation Methods

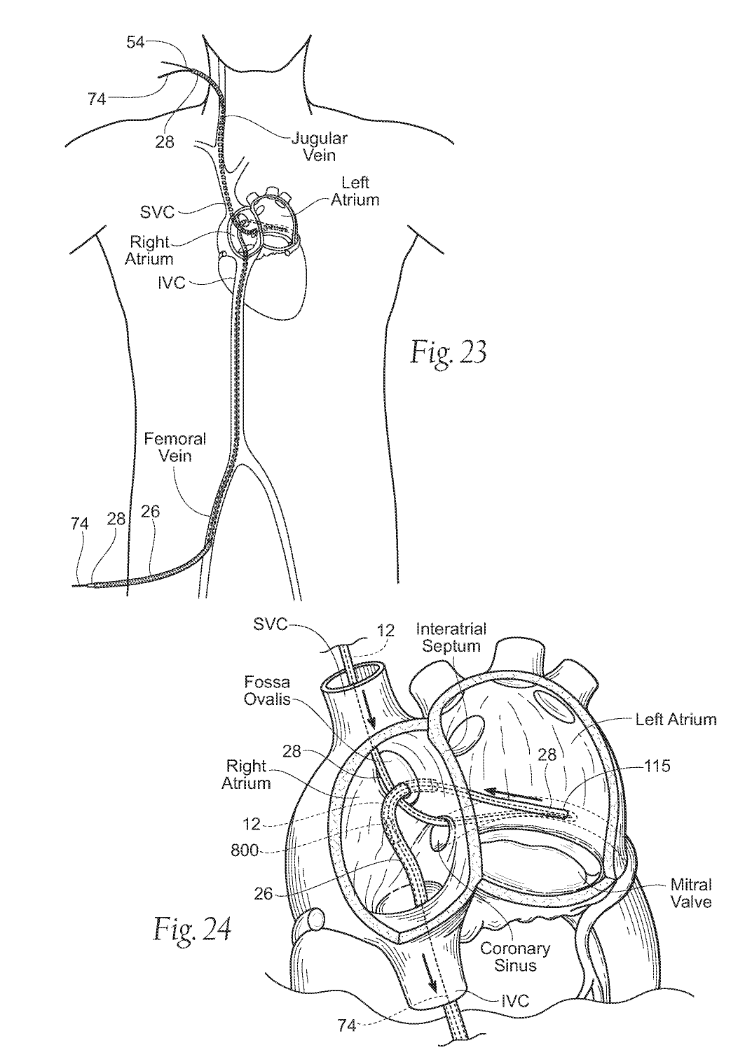

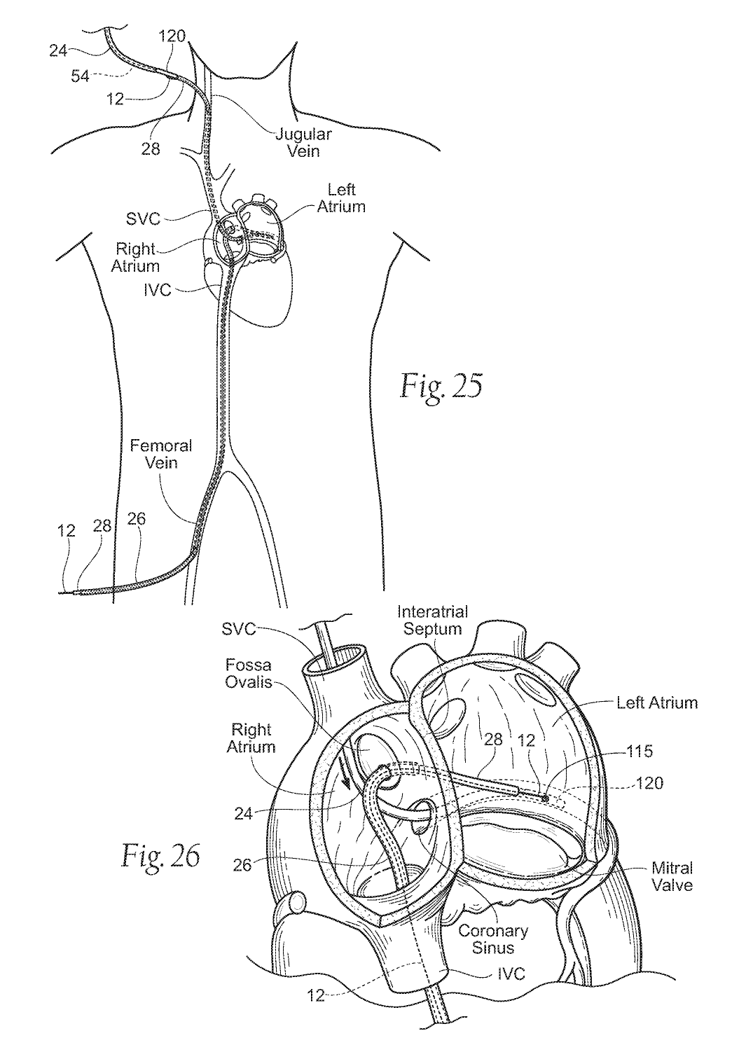

The steps of implantation as previously described can readily accommodate accessing the heart for the septal/sinus shortening procedure using the superior vena cava (SVC), i.e., through the jugular vein, and/or the groin area, i.e., the femoral vein. Access to the vascular system is commonly provided through the use of introducers known in the art. A 16F or less hemostasis introducer sheath (not shown), for example, may be first positioned in the superior vena cava (SVC) through the jugular vein, providing access for the GCV catheter 40. Alternatively, the introducer may be positioned in the subclavian vein. A second 16F or less introducer sheath (not shown) may then be positioned in the groin, i.e., right or left femoral vein, providing access for the LA catheter 60. Access at both the SVC and the right femoral vein, for example, also allows the implantation methods to utilize a loop guide wire. For instance, in a procedure to be described later, a loop guide wire is generated by advancing the LA guide wire 74 through the vasculature until it exits the body and extends external the body at both the superior vena cava sheath and femoral sheath. The LA guide wire 74 may follow an intravascular path that extends at least from the superior vena cava sheath through the interatrial septum into the left atrium and from the left atrium through atrial tissue and through a great cardiac vein to the femoral sheath. The loop guide wire enables the physician to both push and pull devices into the vasculature during the implantation procedure (see FIGS. 23 through 26).

An optional step may include the positioning of a catheter or catheters within the vascular system to provide baseline measurements. An AcuNav™ intracardiac echocardiography (ICE) catheter (not shown), or similar device, may be positioned via the right femoral artery or vein to provide measurements such as, by way of non-limiting examples, a baseline septal-lateral (S-L) separation distance measurement, atrial wall separation, and a mitral regurgitation measurement. Additionally, the ICE catheter may be used to evaluate aortic, tricuspid, and pulmonary valves, IVC, SVC, pulmonary veins, and left atrium access.

Some clinicians may seek to avoid SVC access through a neck region for various reasons, e.g., the typical placement of fluoroscopic equipment near the upper torso, or working near the head of an awake patient. In this situation, the clinician may prefer access only through the groin.

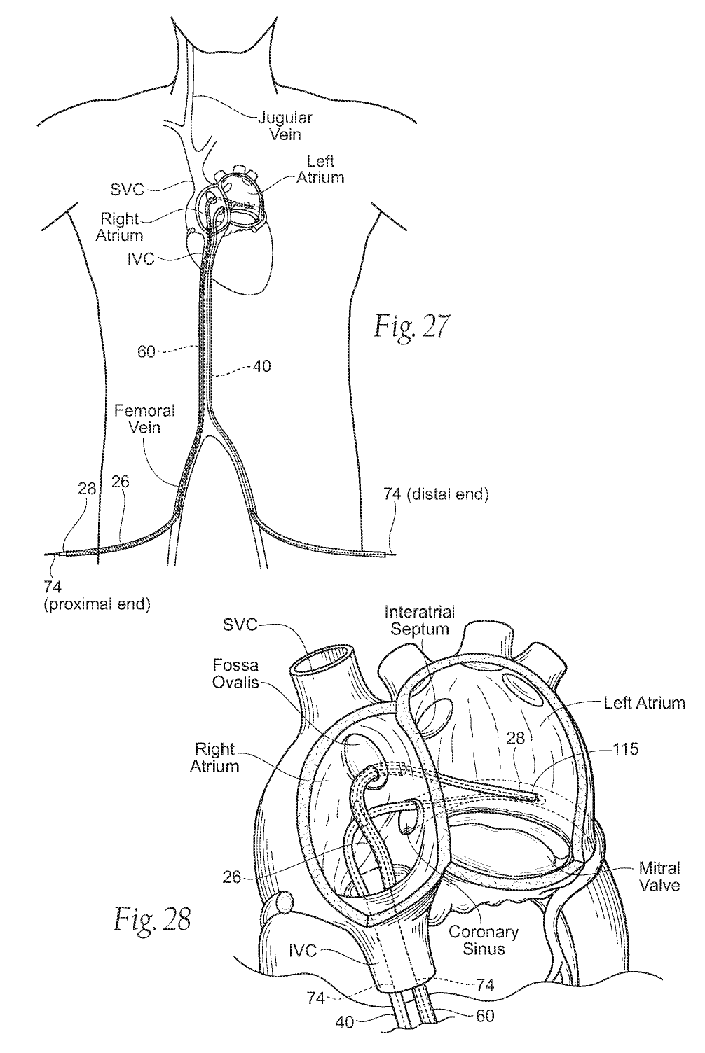

As shown in FIGS. 27 and 28, the proximal end of a single guide wire 74 can be passed through the LA catheter 60, which gains access through the right femoral vein into the right atrium through the IVC, and from there into the left atrium through the septum. The guide wire 74 exits into the GVC catheter 40 in the GVC in the left atrium, for passage through the GVC catheter 40, which gains access through the left femoral vein into the right atrium through the IVC, and from there into the GVC through the coronary sinus. The distal end of the guide wire 74 therefore exits from the left femoral vein. In this arrangement, when access is achieved through a femoral vein, the GVC catheter 40 requires a greater length than when access is achieved through the neck. Further, the GCV catheter 40 desirably includes a more “cane-like” shape at its distal end to make a bend into the coronary sinus.

Alternatively, since the femoral vein is relatively large, it is possible to place both LA catheter 60 and GVC catheter 60 in a single large hemostasis sheath in one vein (left or right), thus avoid two access locations.

In like fashion, a single access point procedure from the neck can be used, which has an advantage of a shorter access distance for the LA catheter 60.

B. Establish Trans-Septal Bridging Element

Now that the posterior bridge stop region 14 has been established, the trans-septal bridging element 12 is positioned to extend from the posterior bridge stop region 14 in a posterior to anterior direction across the left atrium and to the anterior bridge stop region 16.

The bridging element 12 may be composed of a suture material or suture equivalent known in the art. Common examples may include, but are not limited to, 1-0, 2-0, and 3-0 polyester suture, stainless steel braid (e.g., 0.022 inch diameter), and NiTi wire (e.g., 0.008 inch diameter). Alternatively, the bridging element 12 may be composed of biological tissue such as bovine, equine or porcine pericardium, or preserved mammalian tissue, preferably in a gluteraldehyde fixed condition. Alternatively the bridging element 12 may be encased by pericardium, or polyester fabric or equivalent.

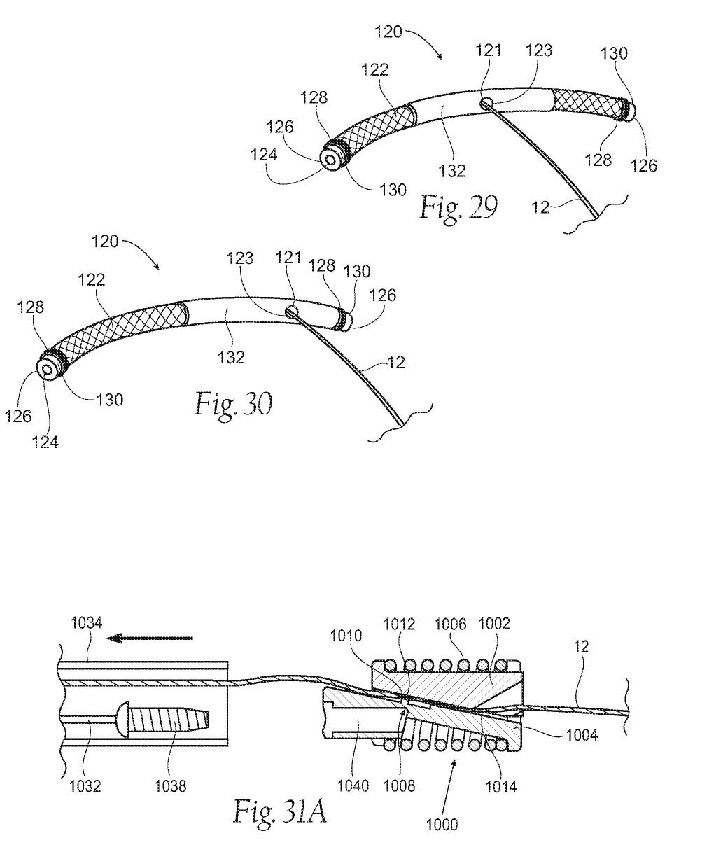

A bridge stop, such as a T-shaped bridge stop 120 is preferably connected to the predetermined length of the bridging element 12. The bridging element 12 may be secured to the T-shaped bridge stop 120 through the use of a bridge stop 20, or may be connected to the T-shaped bridge stop 120 by securing means 121, such as tying, welding, or gluing, or any combination thereof. As seen in FIGS. 29 and 30, the T-shaped bridge stop 120 may be symmetrically shaped or asymmetrically shaped, may be curved or straight, and preferably includes a flexible tube 122 having a predetermined length, e.g., three to eight centimeters, and an inner diameter 124 sized to allow at least a guide wire to pass through. The tube 122 is preferably braided, but may be solid as well, and may also be coated with a polymer material. Each end 126 of the tube 122 preferably includes a radio-opaque marker 128 to aid in locating and positioning the T-shaped bridge stop 120. The tube 122 also preferably includes atraumatic ends 130 to protect the vessel walls. The T-shaped bridge stop 120 may be flexurally curved or preshaped so as to generally conform to the curved shape of the great cardiac vein or interatrial septum and be less traumatic to surrounding tissue. The overall shape of the T-shaped bridge stop 120 may be predetermined and based on a number of factors, including, but not limited to the length of the bridge stop, the material composition of the bridge stop, and the loading to be applied to the bridge stop.

A reinforcing center tube 132 may also be included with the T-shaped bridge stop 120. The reinforcing tube 132 may be positioned over the flexible tube 122, as shown, or, alternatively, may be positioned within the flexible tube 122. The reinforcing tube 132 is preferably solid, but may be braided as well, and may be shorter in length, e.g., one centimeter, than the flexible tube 122. The reinforcing center tube 132 adds stiffness to the T-shaped bridge stop 120 and aids in preventing egress of the T-shaped member 120 through the cored or pierced lumen 115 in the great cardiac vein and left atrium wall.

C. Establish Anterior Bridge Stop Region

Now that the trans-septal bridging element 12 is in position, the anterior bridge stop region 16 is next to be established.

In one embodiment, the proximal portion or trailing end of the bridging element 12 extending exterior the body is then threaded through or around an anterior bridge stop, such as the septal member 30. Preferably, the bridging element 12 is passed through the septal member 30 outside of the body nearest its center so that, when later deployed over the fossa ovalis, the bridging element 12 transmits its force to a central point on the septal member 30, thereby reducing twisting or rocking of the septal member. The septal member is advanced over the bridging element 12 in a collapsed configuration through the catheter 26, and is positioned within the right atrium and deployed at the fossa ovalis and in abutment with interatrial septum tissue. The bridging element 12 may then be held in tension by way of a bridge stop 20 (see FIGS. 18 and 19). The anterior bridge stop 20 may be attached to or positioned over the bridging element 12 and advanced with the septal member 30, or alternatively, the bridge stop 20 may be advanced over the bridging element 12 to the right atrium side of the septal member 30 after the septal member has been positioned or deployed. Alternatively, the bridge stop 20 may also be positioned over the LA guide wire 74 and pushed by the deployment catheter 24 into the right atrium. Once in the right atrium, the bridge stop 20 may then be attached to or positioned over the bridging element 12, and the LA guide wire 74 and deployment catheter 24 may then be completely removed from the body.

1. Bridge Stops

As previously described, a bridge stop serves to secure the bridging element 12 at the posterior or anterior bridge stop region 14, 16, or both.

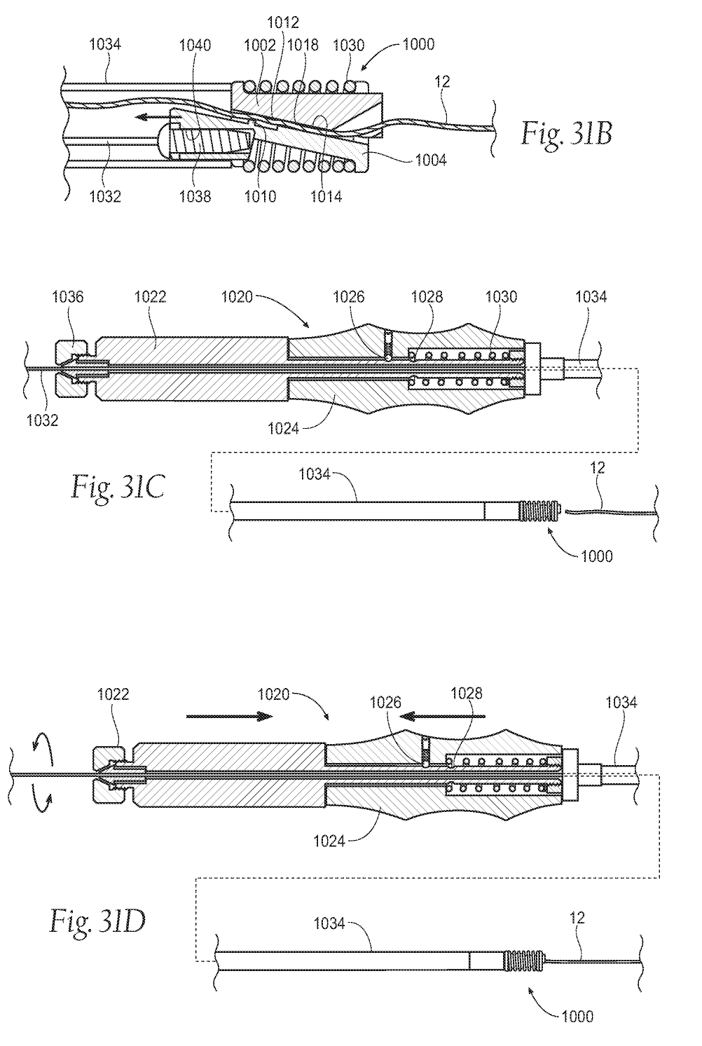

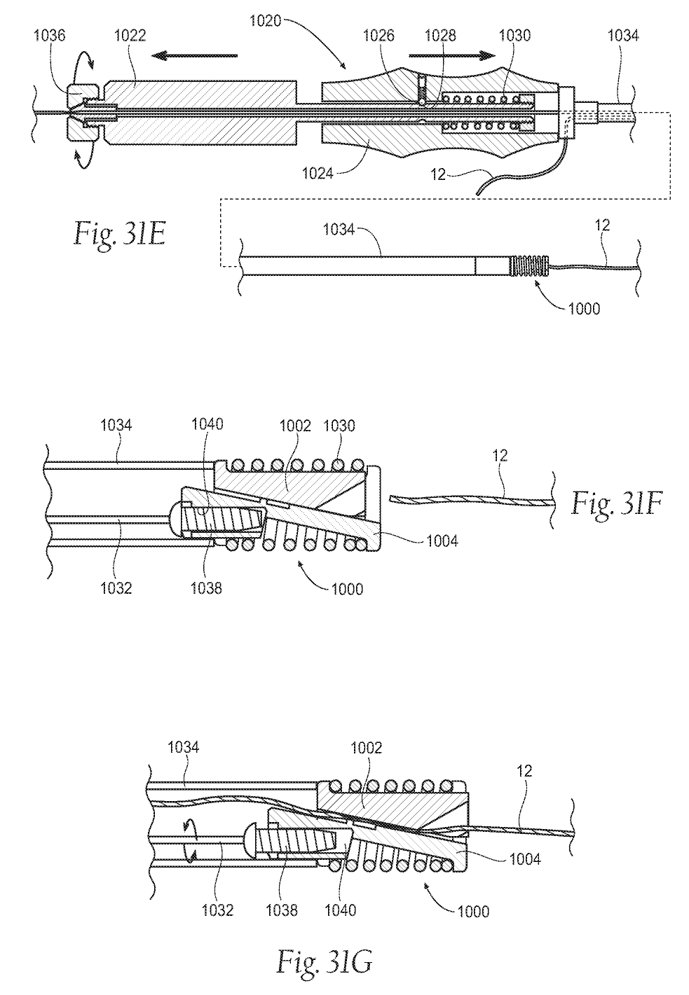

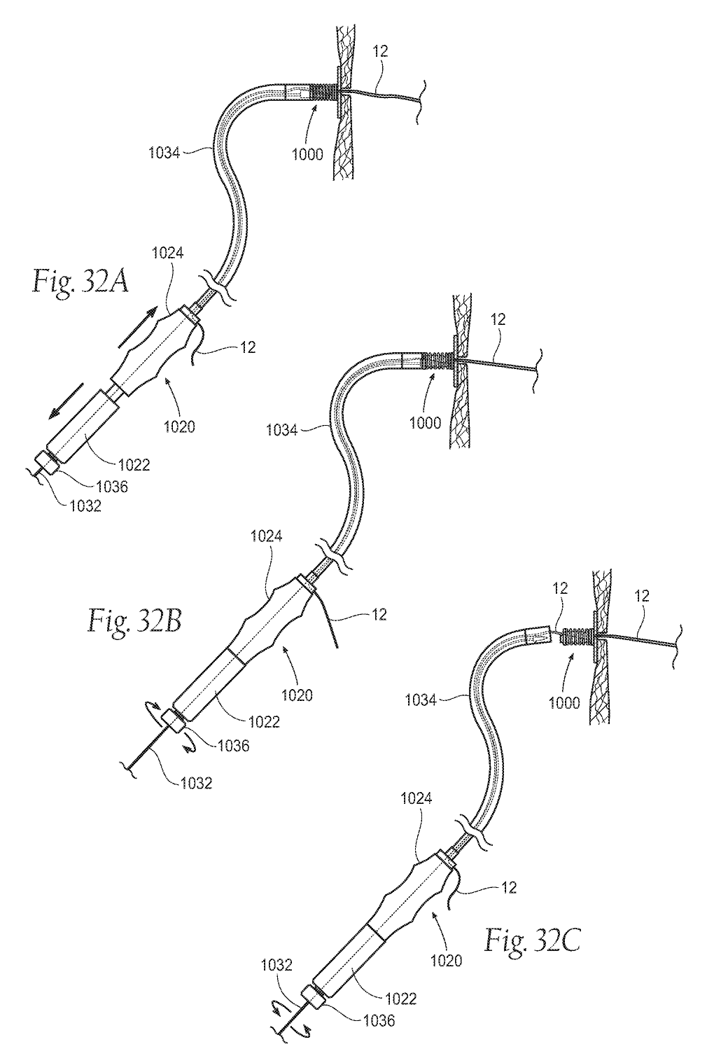

FIG. 31A shows one embodiment of a bridge stop 1000 in accordance with the present invention. FIG. 31A shows the bridge stop 1000 in a closed and locked condition on a bridging element 12. FIG. 32C also shows the bridge stop 1000 in the closed and locked condition, for retaining tension on the bridging element 12.

As shown in FIG. 31A, the bridge stop 1000 includes first and second jaws 1002 and 1004 held together by spring coils 1006. The interior facing surfaces of the jaws 1002 and 1004 defined a passage 1014 between them, which accommodates the bridging element 12. When the spring coils 1006 are in a normal, uncompressed condition, as shown in FIG. 31A, the passage 1014 includes a pinching region 1008, which is defined by the abutment of a pinch arm 1010 on the jaw 1004 against a sloping pinch surface 1012 on the jaw 1002. The pinching region 1008 applies clamping friction to the bridge element 12, preventing relative slippage between the bridge stop 1000 and the bridge element 12. The bridge stop 1000 is in the closed and locked condition, in which the passage 1014 is closed at the pinching region 1018.

As shown in FIG. 31B, the jaws 1002 and 1004 can be moved axially relative to each other, in response to a pulling force on the jaw 1004, concurrent with the application of an opposing force to the jaw 1002. In response to these concurrently applied forces, the spring coils 1006 are compressed, and the pinch arm 1010 moves away from abutment with the sloping pinch surface 1012. The pinching region 1018, and thus the passage 1014, are opened. The bridge stop 1000 in an opened and unlocked condition. When in this opened and unlocked condition, the bridge element 12 can be threaded through the passage 1014, and the bridge stop 1000 can be advanced along the bridge element 12. When the concurrent forces are removed, the spring coils 1006 return to their normally uncompressed condition, moving the jaws 1002 and 1004. The pinch arm 1010 returns back into abutment against the sloping pinch surface 1012. The bridge stop 1000 is again in the closed and locked condition (as shown in FIG. 31A). The passage 1014 is closed at the pinching region 1018, and the pinching region 1008 again applies clamping friction to the bridge element 12.

FIG. 31C shows a bridge stop control device 1020 for operating the bridge stop 1000 between its closed and locked condition and its open and unlocked condition. The bridge stop control device 1020 includes a stationary handle portion 1022 and movable handle portion 1024, and an elongated catheter body 1034 that extends distally from the handle portion 1024.

The handle portions 1022 and 1024 can be moved together and apart, between an adjacent condition (shown in FIGS. 31C and 31D) and an apart condition (shown in FIG. 31E). A spring 1030 normally biases the handle portions toward their adjacent condition (as seen in FIG. 31C).

A spring-biased detent mechanism is carried within the handle portion 1024, comprising a spring loaded ball 1026 that is received with a detent 1028 when the handle portions 1022 and 1024 are in their apart condition (as shown in FIG. 31E). The ball 1026 within the detent 1028 releasably locks the handle portions in their apart condition (as FIG. 31E shows). The frictional locking force between the ball 1026 and detent 1028 yields in response to an external manual force, upon which the spring 1030 brings the handle portions 1022 and 1024 back to their adjacent condition.

The bridge stop control device 1020 includes a control wire 1032 that passes through the handle portion and through the elongated catheter body 1034. A collet assembly 1036 on the handle portion 1022 serves, by rotation, to releasably clamp the control wire 1032. The control wire 1032 includes a screw connector 1038 on its distal end. The screw connector 1038 is sized and configured to threadably engage a receptacle 1040 on the jaw 1004 (as FIGS. 31B and 31F show).

In use, the bridge stop 1000 is coupled to the distal end of the catheter body 1034 by screwing the screw connector 1038 into the jaw receptacle 1040, as shown in FIGS. 31C and 31F. With the bridge stop 1000 in its closed and locked condition (as shown in FIG. 31E), the collet assembly 1036 is rotated to releasably hold tension on the control wire 1032.

The handle portions 1022 and 1024 can then be moved into their apart condition (as shown in FIGS. 31B and 31E). This applies concurrent force upon the jaws 1002 and 1004 of the bridge stop 1000, sliding the jaws apart and placing the bridge stop 1000 in its open and unlocked condition (as shown in FIG. 31B). The bridge element 12 can be threaded through the bridge stop 1000, and passed through the catheter body 1034 through an aperture 1042 near the movable handle portion 1024 (see FIG. 31E). In this condition (see FIGS. 32A and 32B), the bridge stop control device 1020 can be manipulated to slide the bridge stop 1000 along the bridge element 12 to a desired location on the bridge element 12.

Once the desired degree of tension is placed on the bridge element 12 (by pulling on the bridge element 12 with the bridge stop 1000 unlocked and open), the bridge stop 1000 is placed in its closed and locked condition (see FIG. 32B), by rotating the collect assembly 1036 to free tension from the control wire 1032. The tension placed on the bridge element 12 is thereby retained. The handle portions 1022 and 1024 can also be returned to their adjacent position at this time.

As shown in FIG. 32C, the bridge stop control device 1020 is released from the bridge stop 1000 by rotating the control wire 1032. Rotation of the control wire 1032 unthreads the screw connector 1038 from the receptacle 1040 on the jaw 1004 (as is also shown in FIGS. 31A, 31D, and 31G), allowing separation of the bridge stop control device 1020 from the bridge stop 1000.

It is to be appreciated that alternative embodiments of the bridge stop may be configured to have a bridge securing configuration in its static state, so as to require a positive actuation force necessary to allow the bridging element to move freely within or around the bridge stop. When a desirable tension in the bridge element is achieved, the actuation force is removed, thereby returning the bridge stop back to its static state and securing the bridge stop to the bridging element. Alternatively, the bridge stop may be configured to allow free movement of the bridging element 12 in its static state, thereby requiring a positive securing force to be maintained on the bridge stop necessary to secure the bridging element within the bridge stop.

Preferably, the bridge securing feature is unambiguous via tactile or fluoroscopic feedback. The securing function preferably may be locked and unlocked several times, thereby allowing the bridging element to be readjusted. The bridge stop material is also desirably radio-opaque or incorporates radio-opaque features to enable the bridge stop to be located with fluoroscopy.

Further details of representative embodiments of bridge stops can be found in co-pending, commonly owned U.S. patent application Ser. No. 11/389,819, filed Mar. 27, 2006, and entitled “Devices, Systems, and Methods for Reshaping a Heart Valve Annulus,” which is incorporated herein by reference.

D. Bridging Element Adjustment

The anterior bridge stop 20 is preferably positioned in an abutting relationship to the septal member 30, or optionally may be positioned over the septal member hub 31. The bridge stop 20 serves to adjustably stop or hold the bridging element 12 in a tensioned state to achieve proper therapeutic effects.

With the posterior bridge stop region 14, bridging element 12, and anterior bridge stop region 16 configured as previously described, a tension may be applied to the bridging element 12, either external to the body at the proximal portion of the bridging element 12, or internally, including within the vasculature structure and the heart structure. After first putting tension on the bridging element 12, the implant 10 and associated regions may be allowed to settle for a predetermined amount of time, e.g., five seconds. The mitral valve and its associated mitral valve regurgitation are then observed for desired therapeutic effects. The tension on the bridging element 12 may be repeatably adjusted following these steps until a desired result is achieved. The bridge stop 20 is then allowed to secure the desired tension of the bridging element 12. The bridging element 12 may then be cut or detached at a predetermined distance away from the bridge stop 20, e.g., zero to three centimeters into the right atrium. The remaining length of bridging element 12 may then be removed from the vasculature structure.

Alternatively, the bridging element 12 may be allowed to extend into the IVC and into the femoral vein, possibly extending all the way to the femoral access point. Allowing the bridging element to extend into the IVC and into the femoral vein would allow for retrieval of the bridging element in the future, for example, if adjustment of the bridging element is necessary or desired.

The bridging element adjustment procedure as just described including the steps of placing a tension, waiting, observing, and readjusting if necessary is preferred over a procedure including adjusting while at the same time—or real-time—observing and adjusting, such as where a physician places a tension while at the same time observes a real-time ultrasound image and continues to adjust based on the real-time ultrasound image. The waiting step is beneficial because it allows for the heart and the implant to go through a quiescent period. This quiescent period allows the heart and implant to settle down and allows the tension forces and devices in the posterior and anterior bridge stop regions to begin to reach an equilibrium state. The desired results are better maintained when the heart and implant are allowed to settle prior to securing the tension compared to when the mitral valve is viewed and tension adjusted real-time with no settle time provided before securing the tension.

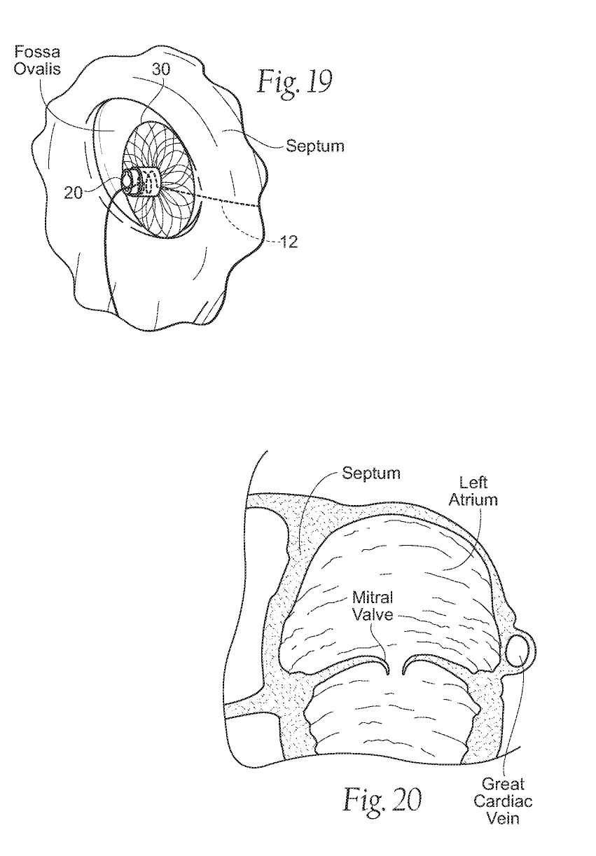

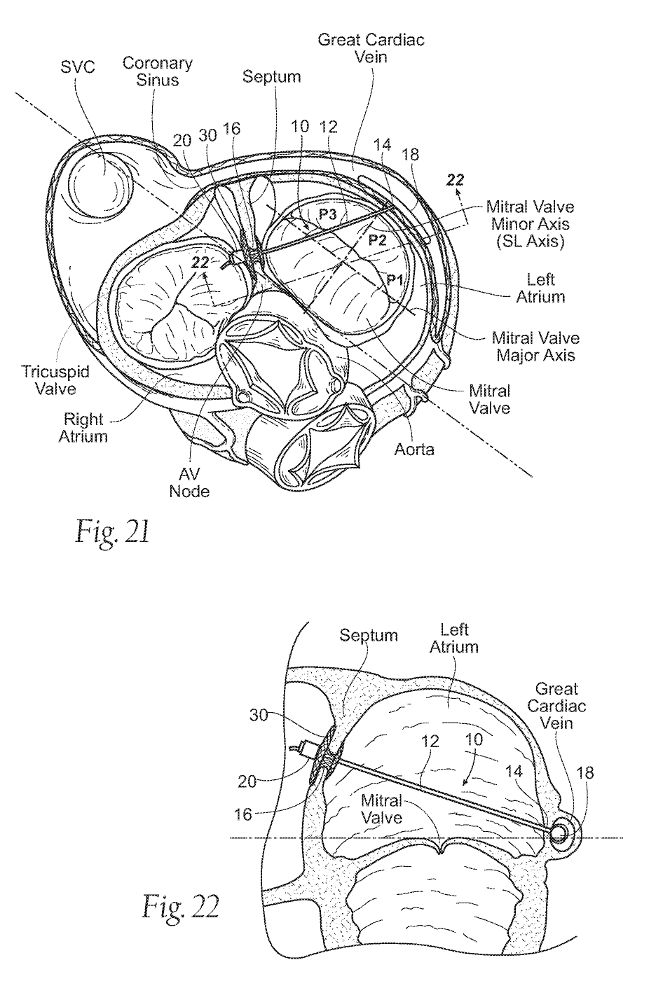

FIG. 20 shows an anatomical view of mitral valve dysfunction prior to the implantation of the implant 10. As can be seen, the two leaflets are not coapting, and as a result the undesirable back flow of blood from the left ventricle into the left atrium can occur. After the implant 10 has been implanted as just described, the implant 10 serves to shorten the minor axis of the annulus, thereby allowing the two leaflets to coapt and reducing the undesirable mitral regurgitation (see FIGS. 21 and 22). As can be seen, the implant 10 is positioned within the heart, including the bridging element 12 that spans the mitral valve annulus, the anterior bridge stop 20 and septal member 30 on or near the fossa ovalis, and the posterior bridge stop 18 within the great cardiac vein.

Further details of representative embodiments of the deployment of an implant 10 of the types shown in FIGS. 10A to 10C by a percutaneous, catheter-based procedure, under image guidance can be found in co-pending, commonly owned U.S. patent application Ser. No. 11/389,819, filed Mar. 27, 2006, and entitled “Devices, Systems, and Methods for Reshaping a Heart Valve Annulus,” which is incorporated herein by reference.

### III. Reduction in Mitral Annular Minor Axis Dimension

With Minor Axis Force Projection

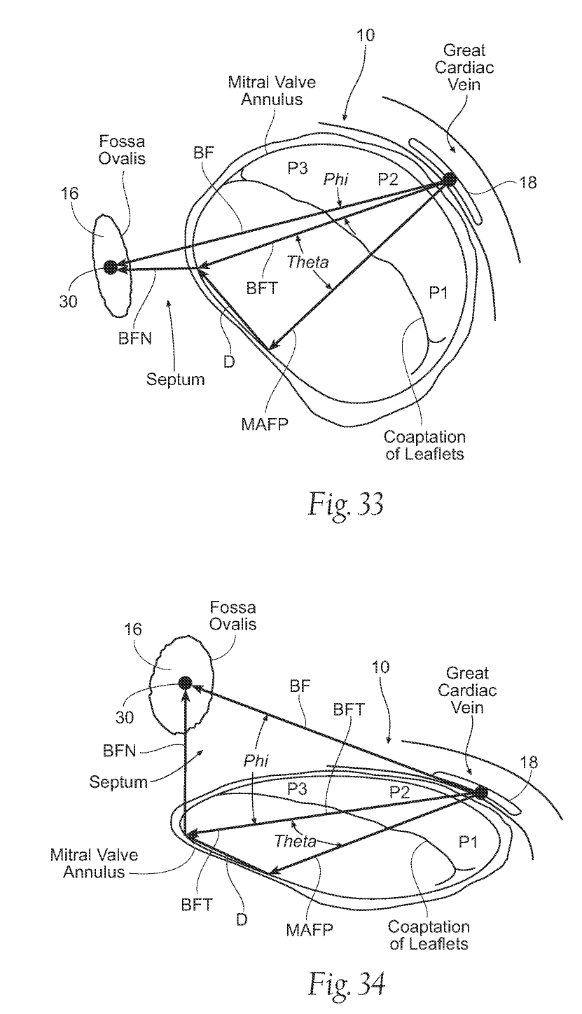

The systems and methods described herein provide a system 10 to shorten the mitral valve minor axis dimension (SL Axis in FIG. 21) of the annulus by applying a selected force vector or projection adapted to reduce/eliminate mitral valve regurgitation. This force vector, described herein as the minor axis force projection MAFP, which acts along the SL axis, is shown in FIGS. 33 and 34. A number of factors are combined to produce a minor axis force projection MAFP adapted for successful reduction in the minor axis dimension. The systems and methods create an upward (lifting) force and an inward (pulling) force acting on, and thus may provide a therapeutic effect to, the ventricular wall of the heart. There is also an equal but opposite downward and inward force acting on the anterior bridge stop region. It is to be appreciated that these descriptions are based on an idealized anatomy, and that anatomies may be not be uniform, and that disease may alter anatomies in different ways.

The magnitude of the minor axis force projection MAFP can be computed from the angles of the bridging element 12 relative to the posterior and anterior bridge stop regions (e.g., the great cardiac vein 14 and the fossa ovalis 16), which include the lateral angle theta, and the superior angle phi, and the bridge force BF, e.g., the tension applied to the bridge.

A. Bridging Element Angle

It should be understood that bridge force BF may be represented as a vector acting along the line of the bridge element 12, i.e., the bridge force vector BF as shown in FIGS. 33 and 34. The direction of vector BF may be described by two components: a lateral angle theta and a superior angle phi (see FIGS. 33 and 34). The lateral angle theta can be described as the angle between the force vector MAFP and the force vector BET, where BFT is defined as the perpendicular projection of vector BE onto a plane parallel to the mitral valve plane and passing through posterior bridge stop 18. The superior angle phi can be described as the angle between the bridge force vector BE, and the force vector BET. The magnitude of vector BFT is equal to the product (magnitude of vector BF x COS phi) with the vertex of the superior angle phi being the posterior bridge stop 18 positioned within the great cardiac vein. The superior angle phi can best be seen in FIG. 34, showing generally a lateral view of the system 10. The magnitude of vector MAFP is equal to the product (magnitude of vector BET x COS theta). The vertex of the lateral angle theta also is the posterior bridge stop 18 positioned within the great cardiac vein, generally in a position behind the P2 posterior mitral leaflet, although the posterior bridge stop 18 may also be positioned generally in a position behind the P1 or P3 posterior mitral leaflet as well. The lateral angle theta can best be seen in FIG. 33, showing generally a superior view of the system 10.

The lateral angle theta may range between about ten degrees to about sixty degrees, and more specifically between about fifteen degrees to about forty degrees, and even more specifically between about twenty degrees to about thirty degrees, and most desirably about twenty-five degrees.

The superior angle phi may range between about zero degrees to about forty-five degrees, and more specifically between about ten degrees to about thirty degrees, and even more specifically between about fifteen degrees to about twenty degrees, and most desirably about seventeen degrees.

The lateral angle theta may offer slightly more variation in range than the superior angle phi due to the anatomical limitations of bridge stop sites able to be used to produce the desired minor axis shortening. For example, the posterior bridge stop 18 is shown located in the great cardiac vein. This site provides a desirable site for the location of a bridge stop due to its accessibility, its viewability under image guidance, and its anatomical relationship to the mitral valve annulus. A portion of the great cardiac vein runs generally along the outer posterior wall of the left atrium. This portion runs in a generally horizontal to a slightly inferior direction as it wraps around the left atrial posterior wall. This portion of the great cardiac vein offers approximately a two centimeter variation in superior to inferior positions for the posterior bridge stop, limiting the range of the superior angle phi, while providing a greater range of the lateral angle theta, i.e., with the posterior bridge stop 18 positioned generally behind the P1, P2, or P3 posterior leaflet.

The anterior bridge stop region 16 may be located in the septal wall, generally at or near the fossa ovalis. This is also a desirable site for the location of a bridge stop due to its accessibility, its viewability under image guidance, and its anatomical relationship to the mitral valve annulus. The septal wall, and more specifically the fossa ovalis, provides minor variation in superior to inferior, and anterior to posterior positions for the anterior bridge stop, limiting the range of both the superior (phi) and lateral (theta) angles. The AV node, the aortic wall, and the posterior atrial wall all provide a boundary for the anterior bridge stop region 16, i.e., positioning the anterior bridge stop.

In addition, as the ranges of both the lateral and superior angles move away from the desired ranges as described above, the beneficial minor axis shortening effects of the implant 10 diminish. For example, as previously described, a desired range of angles for the lateral angle theta is about ten degrees to about sixty degrees. While the implant system 10 is within this range along with the desired ranges for the superior angle phi and the desired bridge force, (i.e., tension) minor axis shortening associated with an upward and inward force acting on the ventricular wall may be achieved. When the lateral angle theta is more than sixty degrees, the implant 10 is less able to affect the minor axis shortening. A lateral angle theta less than ten degrees may not be achievable because of potential anatomical constraints, e.g., an achievable anterior bridge stop region 16 in the septum may be limited by the AV node and/or the aortic wall. More than sixty degrees may be less desirable because as the lateral angle theta increases, the implant 10 may become misaligned with the mitral valve minor axis, creating a force that may no longer be generally aligned with the minor axis. Anatomical constraints may also limit the posterior bridge stop region 14, e.g., within the GCV.

The same limitations apply for the superior angle phi. For example, as previously described, a desired range of angles for the superior angle phi is about zero degrees to about forty-five degrees. When the implant system 10 is within this range along with the desired ranges for the lateral angle theta and the desired bridge force, minor axis shortening may be achieved. A value of less than zero degrees for the superior angle phi is expected to be rare in humans because the fossa ovalis is usually higher above the mitral valve plane than the GCV. When the superior angle phi is more than forty-five degrees, the implant 10 is less able to affect the minor axis shortening. This is because as the superior angle phi increases, the ability of the implant 10 to produce a pulling force (inward) in coordination with a lifting force (upward) acting on the ventricular wall may be reduced, e.g., the system 10 may provide a lifting force with minimal or no inward force. Anatomical constraints may also limit the posterior bridge stop region, e.g., within the GCV.

B. Bridge Force

A second factor is the magnitude of the bridge force vector BF acting through the bridging element 12 of the system 10. As previously described, the bridge force vector BF (i.e., tension) is generated by the bridging element, allowing the system 10 to shorten the minor axis dimension and also causes a focal inward and upward traction to be applied to the posterior bridge stop in the direction of the anterior bridge stop.

The magnitude of bridge force vector BF may be measured as a force, with the unit of measure being the pound-force (lbf). The magnitude of bridge force vector BF may range between about 0.1 lbf to about 1.6 lbf, and more specifically between about 0.2 lbf to about 1.0 lbf.

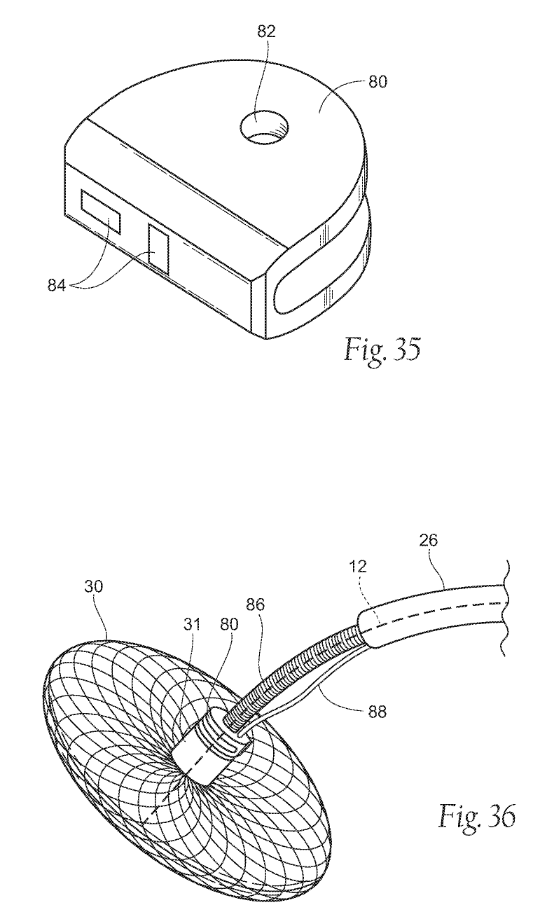

During implantation of the system 10, a load cell or similar force sensing means 80 may be used to provide a measure of the magnitude of bridge force vector BF. As can be seen in FIG. 35, the load cell 80 includes an aperture 82 adapted to allow the load cell 80 to be positioned over the bridging element 12. One or more strain gages 84 are electrically coupled to a measurement device (not shown) by way of a wire cable 88 extending from the load cell 80 and through the catheter 26 to the measurement device.

As can be seen in FIG. 36, the load cell 80 may be positioned between the anterior bridge stop 30 and a stacked coil tube or spring 86 coupled to the distal end of the catheter 26. As shown, the load cell 80 is positioned between the hub 31 of the anterior bridge stop 30 and the stacked coil spring 86, with the bridging element 12 extending through the anterior bridge stop 30, through the aperture 82 in the load cell 80, through the stacked coil spring 86, and through the catheter 26 and extending exterior the body.

When the bridging element 12 is ready to be adjusted to a desired tension, the bridging element (desirably accessible exterior the body) is tensioned (pulled) while simultaneously the catheter 26 and associated stacked coil spring 86 is compressed (pushed). The stacked coil spring 86 provides a highly flexural compliant member adapted to minimize a force artifact on the load cell 80 caused by the catheter 26. The stacked coil spring 86 may be similar to a coiled guide wire, and provides desired pushability and trackability characteristics. Tension and compression become equal when the bridging element 12 is affixed to the hub (proximal end) of the catheter 26. It is essential that there be no net force by the catheter 26 on the anterior bridge stop 30. Under these conditions, the compression force measured by the load cell 80 will be equal to the bridge tension, i.e., the magnitude of the force vector BF.

C. Minor Axis Force Projection Determination

As described above, the minor axis force projection MAFP may be determined from the lateral angle theta, the vertical angle phi, and the magnitude of the bridge force vector BF applied to the bridging element 12. A range of these elements may be combined to produce a minor axis force projection MAFP magnitude ranging from about 0.04 lbf to about 1.58 lbf for creating inward tractions that achieve a successful reduction in the minor axis dimension.

As previously described, the system 10 produces a bridging element 12 under tension. The bridging element 12 imposes an upward (lifting) and inward (pulling) force(s) on the posterior bridge stop 18 which is represented by the force vector bridge force BF, with the force vector BF having a direction and a magnitude. Conceptually, there is also an equal and opposite force vector (i.e., −BF) acting on the anterior bridge stop 30. The force vector BE may be decomposed into a shortening force tangent component BFT (a vector) and a lifting force normal component BFN (a vector), where vector BF=BET+BEN by vector addition.

The magnitude of BFT may be expressed using the formula: magnitude of vector BFT=magnitude of vector BF×COS phi. Force vector BFT may also be decomposed into two components, that being the minor axis force projection MAFP (a vector), and the force vector D, where vector BFT=MAFP+D by vector addition.

Using the same reasoning, the magnitude of the minor axis force projection MAFP can be expressed using the formula: magnitude of MAFP=magnitude of BFT×COS theta. Knowing the equation for the magnitude of BET, the minor axis force projection MAFP may now be expressed using the formula: magnitude of MAFP=magnitude of BF×COS theta×COS phi.

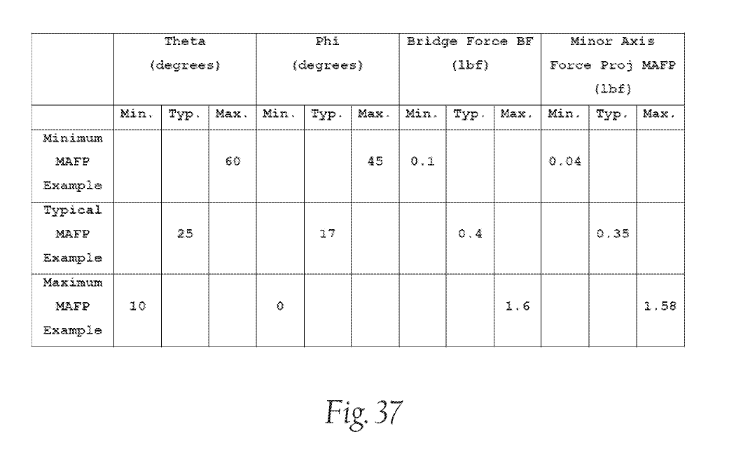

Using this equation, FIG. 37 shows representative ranges of the lateral angle theta, the superior angle phi, and the measured bridge force magnitude BF, that when combined produce a minimum, typical, and maximum magnitude of the minor axis force projection MAFP.

As can be seen, the range of the minor axis force projection MAFP values that are possible across the range of inputs, (i.e., lateral angle theta, superior angle phi, and bridge force BF) is between about 0.04 lbf and about 1.58 lbf. The typical minor axis force projection MAFP magnitude is about 0.35 lbf, resulting from a desired bridge force BE magnitude of about 0.4 lbf, with a lateral angle theta of about 25 degrees, and a superior angle phi of about 17 degrees. Note that in the maximum MAFP magnitude example, the maximum bridge force BF magnitude of about 1.6 lbf is near to the maximum minor axis force projection MAFP magnitude of about 1.58. This is because the minor axis force projection MAFP is determined using generally small values of the lateral angle theta (ten degrees) and the superior angle phi (zero degrees).

D. Experimental Data

Experimental data from a 3, 6, and 12 month ovine model trial of the implant system 10 has shown an overall sustained reduction in the minor axis dimension. The systolic septal lateral dimension was 26.3±1.6 mm pre-implant, 21.7±1.5 mm post-implant (mean 17.4 percent reduction), and 21.6±2.2 mm at a latest follow-up. Mean forces (i.e., magnitude of bridge force vector BF) exerted on the bridging element 12 in vivo were measured and ranged from about 0.26 lbf to about 0.42 lbf to achieve an minor axis dimension reduction of up to about 30 percent.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method comprising:
providing an implant adapted for spanning a heart valve,
extending the implant through a first heart chamber and to second heart chamber, and
applying a tension to the implant, the tension creating a force ranging between about 0.1 lbf to about 1.6 lbf.

2. A method according to claim 1:
further including measuring the tension on the implant, the measuring comprising:
providing a catheter including a flexural compliant member at a distal end,
providing force sensing means coupled to a measurement device,
positioning the force sensing means between the implant in at least one of the first heart chamber and the second heart chamber and the flexural compliant member,
applying a tension to the implant while simultaneously pushing the catheter, and
measuring the tension.

3. The method according to claim 1:
wherein extending the implant comprises extending the implant through a right atrium and to a left atrium.

4. The method according to claim 1:
wherein extending the implant comprises extending the implant through a left atrium and to a right atrium.

5. A method according to claim 4:
wherein extending the implant through a left atrium includes extending the implant through a posterior atrial wall.

6. A method according to claim 4:
wherein extending the implant through a left atrium includes extending the implant from a great cardiac vein and through a posterior atrial wall.

7. A method according to claim 4:
wherein extending the implant to the right atrium includes extending the implant through the septum.

8. A method according to claim 4:
wherein extending the implant to the right atrium includes extending the implant through the fossa ovalis.

9. A method according to claim 1:
wherein the generated tension results in an upward force and an inward force acting on a heart wall.

10. A method according to claim 9:
wherein the heart wall is a left ventricular wall.

11. A method according to claim 1:
wherein the generated tension results in a downward force and an inward force acting on a septal wall.

12. A method according to claim 11:
wherein the septal wall is a left atrial wall.

13. A system comprising:
an implant adapted for spanning a heart valve,
the implant adapted to extend through a first heart chamber and to a second heart chamber, and
the implant adapted to generate a tension, the tension creating a force ranging between about 0.1 lbf to about 1.6 lbf.

14. A system for restoring coaptation of a heart valve comprising:
an implant comprising a first angle, a second angle, and a force, and
the first angle, the second angle, and the force being combined to produce a force projection having a range between about 0.04 lbf to about 1.58 lbf.

15. A system according to claim 14:
wherein the first angle comprises a vertex positioned at or near a great cardiac vein.

16. A system according to claim 14:
wherein the first angle comprises a range between about 10 degrees and about 60 degrees.

17. A system according to claim 14:
wherein the second angle comprises a vertex positioned at or near a great cardiac vein.

18. A system according to claim 14:
wherein the second angle comprises a range between about zero degrees and about 45 degrees.

19. A system according to claim 14:
wherein the force comprises a range between about 0.1 lbf to about 1.6 lbf.

20. A system according to claim 14:
wherein the force comprises an upward force and an inward force acting on a heart wall.

21. A system according to claim 20:
wherein the heart wall is a left ventricular wall.

22. A system according to claim 14:
wherein the force comprises a downward force and an inward force acting on a septal wall.

23. A system according to claim 22:
wherein the septal wall is a left atrial wall.

24. A method for restoring coaptation of a heart valve comprising:
providing an implant,
extending the implant through a first heart chamber and to a second heart chamber, the implant comprising a first angle, a second angle, and a force, the first angle, the second angle, and the force combining to produce a force projection having a range from about 0.04 lbf to about 1.58 lbf, and
restoring coaptation of the heart valve.

25. The method according to claim 24:
wherein extending the implant comprises extending the implant through a left atrium and to a right atrium.

26. A method according to claim 24:
wherein the first angle comprises a vertex positioned at or near the great cardiac vein.

27. A method according to claim 24:
wherein the first angle comprises a range between about 10 degrees and about 60 degrees.

28. A method according to claim 24:
wherein the second angle comprises a vertex positioned at or near the great cardiac vein.

29. A method according to claim 24:
wherein the second angle comprises a range between about zero degrees and about 45 degrees.

30. A method according to claim 24:
wherein the force comprises an upward force and an inward force acting on a heart wall.

31. A method according to claim 30:
wherein the heart wall is a left ventricular wall.

32. A method according to claim 24:
wherein the force comprises a downward force and an inward force acting on a septal wall.

33. A method according to claim 32:
wherein the septal wall is a left atrial wall.

34. A method according to claim 24:
wherein the force comprises a range between about 0.1 lbf to about 1.6 lbf.

35. A method of measuring the tension on a heart implant comprising:
providing a catheter including a flexural compliant member at a distal end,
providing force sensing means coupled to a measurement device,
positioning the force sensing means between the implant and the flexural compliant member,
applying a tension to the implant while simultaneously pushing the catheter, and
measuring the tension.

36. A method according to claim 35:
further comprising positioning the force sensing means between the implant in a heart chamber and the flexural compliant member.

37. A method according to claim 35:
wherein the tension results in an upward tension and an inward tension acting on a heart wall.

38. A method according to claim 37:
wherein the heart wall is a left ventricular wall.

39. A method according to claim 35:
wherein the tension results in a downward tension and an inward tension acting on a septal wall.

40. A method according to claim 39:
wherein the septal wall is a left atrial wall.

41. A method according to claim 36:
wherein the heart chamber comprises a left atrium or a right atrium or a left ventricle or a right ventricle.

42. A system comprising:
an implant adapted to extend through a first heart chamber and to a second heart chamber,
a catheter including a flexural compliant member at a distal end,
force sensing means coupled to a measurement device, the force sensing means positioned between the implant and the flexural compliant member,
the implant adapted to generate a tension while the catheter is simultaneously pushed, and
the measurement device adapted to provide a measurement of the tension.
